(19) United States
(12) Patent Application Publication
(10) Pub. No.: US 2005/0049212 A1
(43) Pub. Date: Mar. 3, 2005

(54) Bestrophin and bestrophin homologous proteins involved in the regulation of energy homeostasis

(76) Inventors: Arnd Steuernagel, Gottingen (DE); Gunter Bronner, Gottingen (DE); Rudiger Fritsch, Gottingen (DE); Karsten Eulenberg, Borenden (DE); Thomas Ciossek, Ravensburg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/492,032

(22) PCT Filed: Oct. 9, 2002
(86) PCT No.: PCT/EP02/11321

(30) Foreign Application Priority Data
Oct. 9, 2001 (EP) 01124059.5

(51) Int. Cl. A61K 48/00; C12Q 1/68

(52) U.S. Cl. 514/44; 435/6

(57) ABSTRACT

The present invention discloses Bestrophin homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of diseases and disorders, for example, but not limited to, metabolic diseases such as obesity as well as related disorders such as eating disorder cachexia, diabetes mellitus, hypertension, coronary heart disease, hypercholesterolemia, osteoarthritis, gallstones, cancers of the reproductive organs, and sleep apnea.

This invention relates to the use of nucleic acid and amino acid sequences of Bestrophin and Bestrophin homologous proteins (for example, VMD2, VMD2-like protein 1, VMD2-like protein 2, or VMD2-like protein 3), and to the use of these sequences and effectors thereof in the diagnosis, study, prevention, and treatment of diseases and disorders, for examples but not limited to, metabolic diseases such as obesity, body-weight regulation, thermogenesis as well as related disorders such as eating disorder, cachexia, diabetes mellitus, hypertension, coronary heart disease, hypercholesterolemia, osteoarthritis, gallstones, cancers of the reproductive organs, and sleep apnea.

Obesity is one of the most prevalent metabolic disorders in the world. It is a still poorly understood human disease that becomes more and more relevant for western society. Obesity is defined as an excess of body fat, frequently resulting in a significant impairment of health. Besides severe risks of illness such as diabetes, hypertension and heart disease, individuals suffering from obesity are often isolated socially. Obesity is influenced by genetic, metabolic, biochemical, psychological, and behavioral factors. As such, it is a complex disorder that must be addressed on several fronts to achieve lasting positive clinical outcome. Obese individuals are prone to ailments including: diabetes mellitus, hypertension, coronary heart disease, hypercholesterolemia, osteoarthritis, gallstones, cancers of the reproductive organs, and sleep apnea.

Obesity is not to be considered as a single disorder but a heterogeneous group of conditions with (potential) multiple causes. For example, obesity is also characterized by elevated fasting plasma insulin and an exaggerated insulin response to oral glucose intake (Koltermann, J. Clin. Invest 65, 1980, 1272-1284) and a clear involvement of obesity in type 2 diabetes mellitus can be confirmed (Kopelman, Nature 404, 2000, 635-643).

Even if several candidate genes have been described which are supposed to influence the homeostatic system(s) that regulate body mass/weight, like leptin, VCPI, VCPL, or the peroxisome proliferator-activated receptor (PPAR)-gamma co-activator, the distinct molecular mechanisms and/or molecules influencing obesity or body weight/body mass regulations are not known.

Therefore, the technical problem underlying the present invention was to provide for means and methods for modulating (pathological) metabolic conditions influencing thermogenesis, body-weight regulation and/or energy homeostatic circuits. The solution to said technical problem is achieved by providing the embodiments characterized in the claims.

Accordingly, the present invention relates to a nucleic acid of the Bestrophin gene family, particularly the human Bestrophin genes (for example, VMD2, VMD2-like protein 1, VMD2-like protein 2, or VMD2-like protein 3) having novel functions in body-weight regulation, energy homeostasis, metabolism, and obesity as well as related disorders such as eating disorder, cachexia, diabetes mellitus, hypertension, coronary heart disease, hypercholesterolemia, osteoarthritis, gallstones, cancers of the reproductive organs, and sleep apnea. The nucleic acid, the protein coding therefore and an antibody, aptamer or another receptor recognizing the nucleic acid or the protein may be used for diagnostic or therapeutic purposes or as a target for the development of novel agents.

One gene of the human Bestrophin family (also referred to as VMD2) is expressed in several tissues including the retinal pigment epithelium (RPE) where it is localized at the epithelial basolateral plasma membrane (Petrukhin et al., 1998, Nat Genet 19: 241-247; Marmorstein et al., 2000, Proc Natl Acad Sci USA, 97(23):12758-12763). Heterozygous mutations of the Bestrophin (VDM2) gene are associated with Best macular dystrophy (BMD). Best macular dystrophy is a dominantly inherited, early onset disease of macular degeneration that may develop subretinal neovascularisation similar to the wet type of age-related macular degeneration. Macular degeneration is a leading cause of blindness that affects the aged population. Best vitelliform macular dystrophy (VDMZ) is a macular degeneration characterized by the deposition of lipofuscin-like material within and below the RPE and is associated with degeneration of the RPE and overlying photoreceptors (Allikmets, 1999, Hum Genet 104(6):449-453). Bestrophin is the protein identified as being responsible for VDM2 and possibly other forms of maculopathy with phenotypic characteristics similar to Best disease.

Structural computer analysis of the Bestrophin gene sequence suggest that the encoded peptide is a transmembrane protein that defines a new family of anion, in particular chloride channels (ion exchangers) (Marmorstein A D. et al. 2000, supra; Gomez A. et al. 2001, DNA Seq 12(5-6):431-435; Sun et al., 2002, PNAS 99(6), 4008-4013). Bestrophins from different species can form oligomeric chloride (anion, nitrate, bicarbonate) channels responsible for the calcium sensitive chloride conductance in RPE cells. Loss of chloride conductance can also impair the co-transport of nutrients and other essential molecules in RPE which leads to the observed degeneration of RPE, including accumulation of lipofuscin-like material within and below the RPE (Sun H. et al., 2002, supra).

It has recently been shown that Bestrophin interacts physically and functionally with Protein Phosphatase 2A (PP2A) (Marmorstein, 2002, J. Biol. Chem. 277(34), 30591-30597). The beta catalytic subunit of PP2A (PP2Ac) and the structural scaffold subunit of PP2A were immunoprecipitated together with Bestrophin. Bestrophin is phosphorylated in RPE-J cells that is sensitive to the protein phosphatase inhibitor okadaic acid. It was shown that PP2A dephosphorylates Bestrophin in vitro suggesting the regulation of the Bestrophin anion channel activity through PP2A. Therefore, Bestrophin is a member of the signal transduction-pathway that modulates the light peak in the eye (Marmorstein et al. 2002, supra).

Recently, Bestrophin overexpression was shown to directly influence the chloride conductance of HEK293 cells, which led the authors conclude to assign chloride channel activity to bestrophin and thereby to suggest VMD as channelopathy (Sun et al., 2002, supra). Furthermore, the physical interaction between bestrophin and a protein phosphatase, PP2A, suggests that phosphorylation or dephosphorylation of bestrophin may act as the on/off switch for the light peak or modulate its amplitude or timing (Marmorstein, 2002, supra).

Although a clear function of Bestrophins in Best macular dystrophy has been shown, no function in the regulation of energy homeostasis has been described up to date. Surprisingly, we found that Bestrophins are involved in the regulation of energy homeostasis. A genetic screen was used to identify that mutation of a Bestrophin homologous gene causes obesity, reflected by a significant increase of triglyceride content, the major energy storage substance. In this invention we demonstrate that the correct gene dose of Bestrophin and Bestrophin homologous proteins is essential for maintenance of energy homeostasis. In addition, we found that the expression of Bestrophins is high in the hypothalamus and other brain areas suggesting roles in the regulation of the metabolism. We could clearly see that VDM2-like protein 3 and VDM2 are ubiquitously expressed with high expression in white and brown adipose tissue. We could also see that the expression of VMD2 like protein 3 is downregulated in genetically obese mice and in mice under high fat diet, whereas expression of VMD2 is upregulated in those obese mice. Further, VMD2 expression is upregulated in human preadipocytes.

Based on these results, it can be concluded that polynucleotides encoding proteins with homologies to Bestrophins (for example, VMD2, VMD2-like protein 1, VMD2-like protein 2, or VMD2-like protein 3) present the opportunity to investigate diseases and disorders as described above. Thus, new compositions useful in diagnosis, treatment, and prognosis of metabolic diseases and disorders and related diseases are provided.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure.

The present invention discloses that Bestrophin homologous proteins (for example, VMD2, VMD2-like protein 1, VMD2-like protein 2, or VMD2-like protein 3) encoded by nucleic acids of the Bestrophin gene family are regulating the energy homeostasis and fat metabolism, especially the metabolism and storage of triglycerides. The invention also relates to vectors, host cells, receptors or effectors of the proteins or nucleic acids such as antibodies, aptamers, peptides, antisense molecules, ribozymes, RNAi molecules or low molecular weight inhibitors or activators and recombinant methods for producing the polypeptides and polynucleotides of the invention. The invention also relates to the use of these molecules in the diagnosis, study, prevention, and treatment of diseases and disorders as described above.

The term “GenBank Accession number” relates to NCBI GenBank database entries (Benson et al, Nucleic Acids Res. 28, 2000, 15-18).).

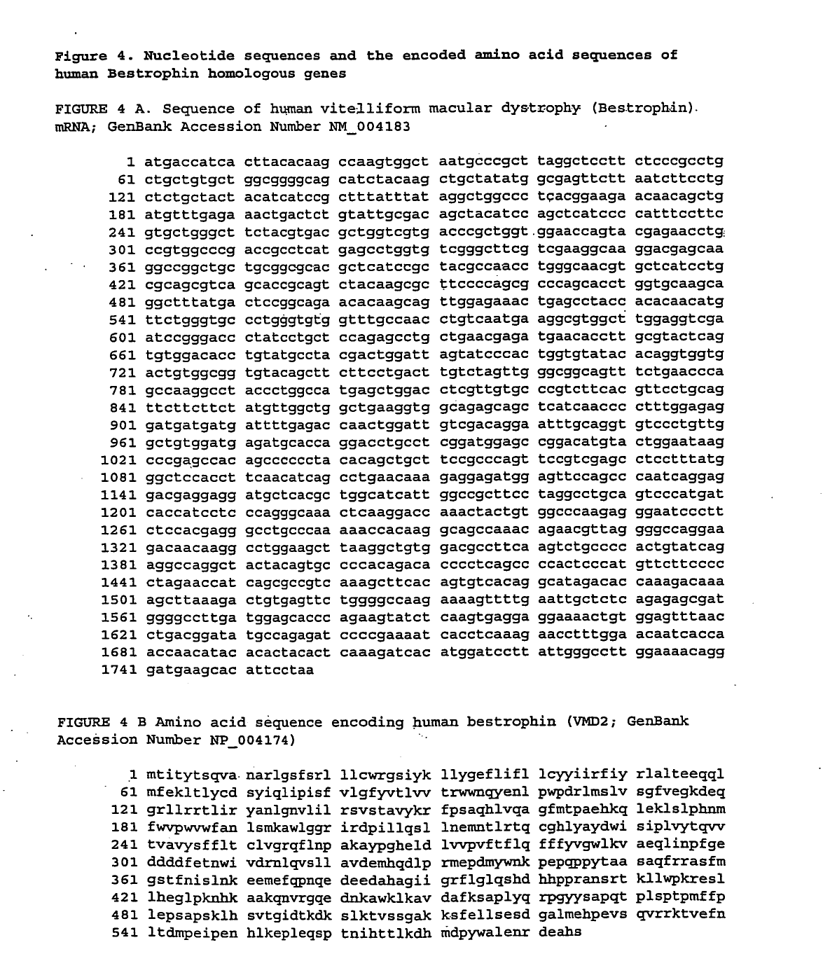

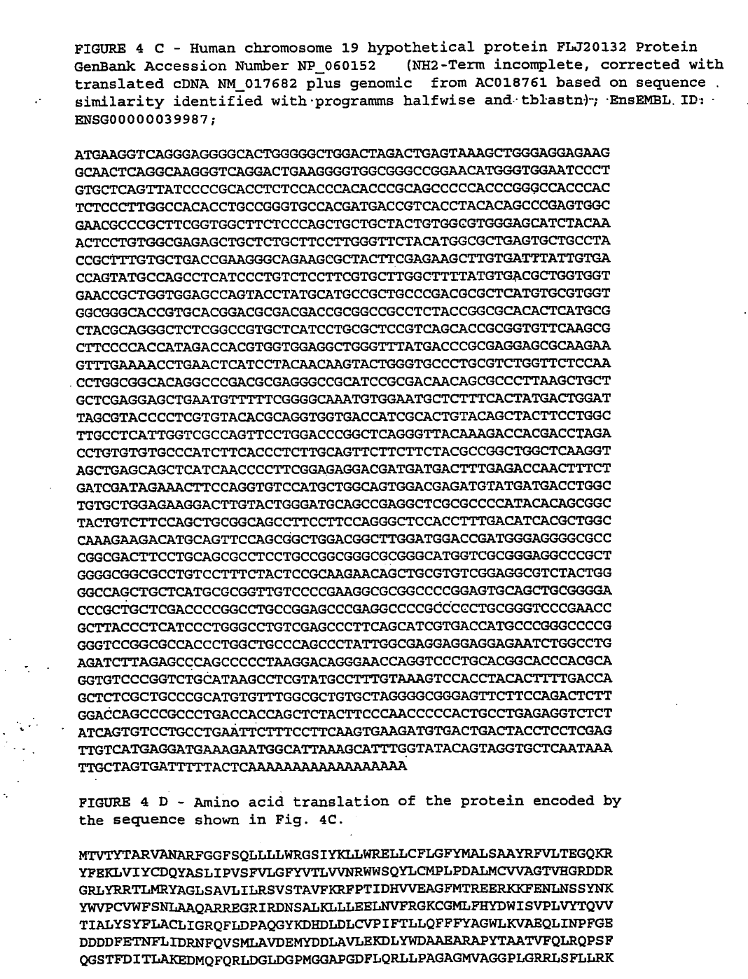

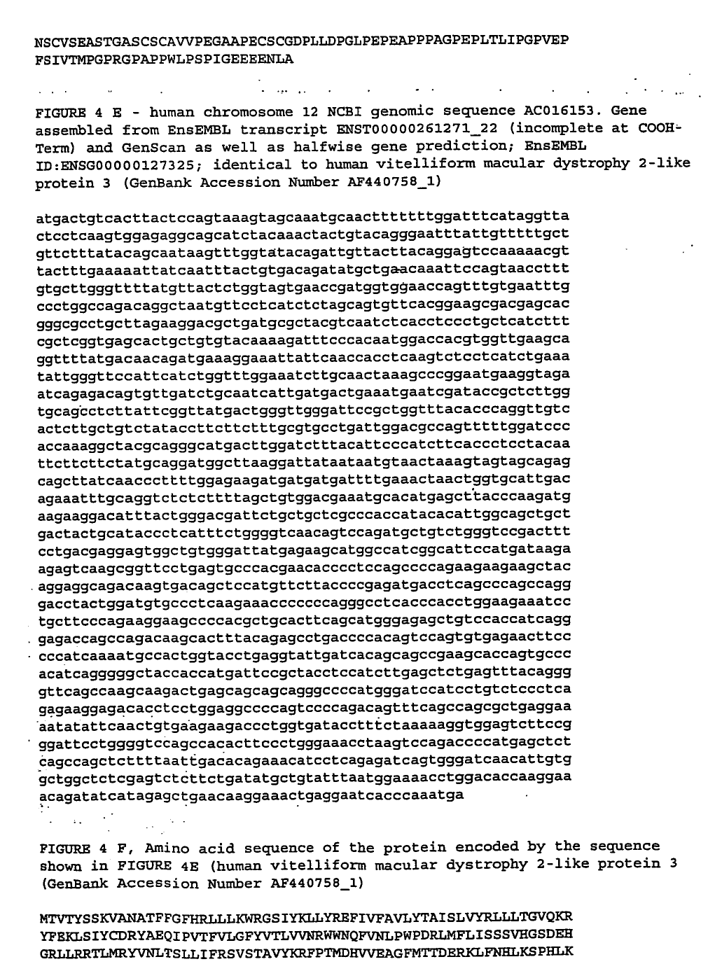

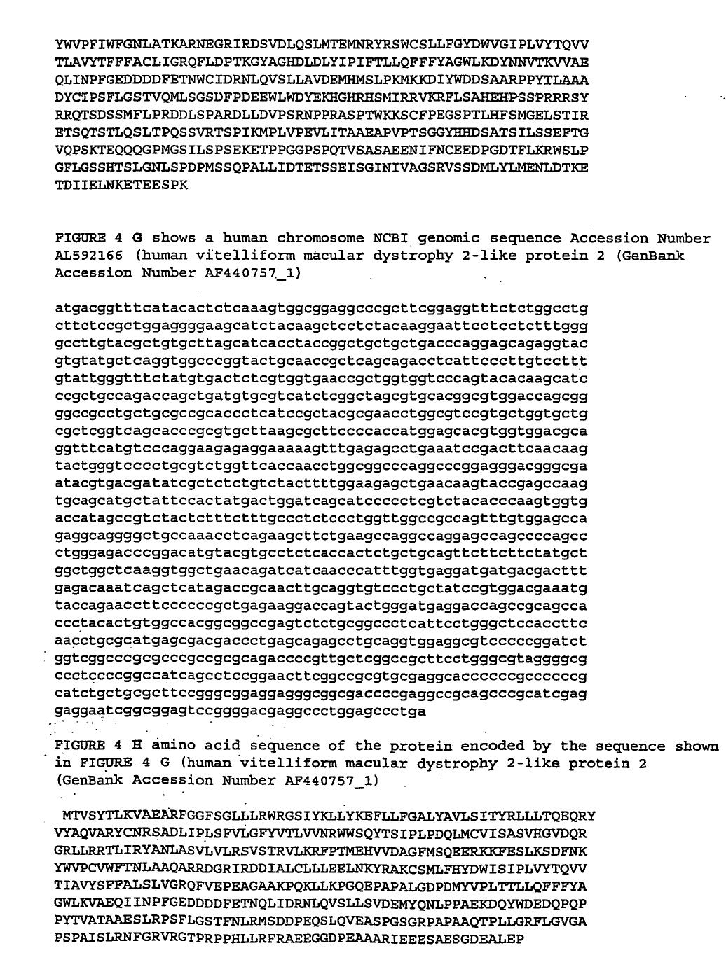

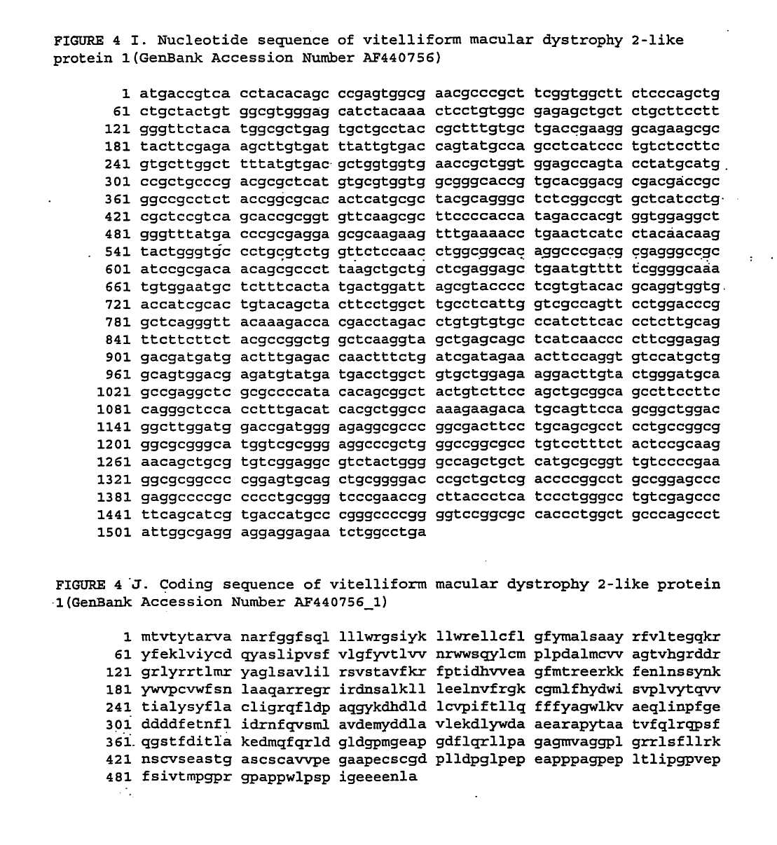

Bestrophin homologous proteins and nucleic acid molecules coding therefore are obtainable from insect or vertebrate species, e.g. mammals or birds. Particularly preferred are human Bestrophin homologous nucleic acids, particularly nucleic acids encoding a
(i) a human Bestrophin protein on chromosome 11 (or also refered to as human vitelliform macular dystrophy, VMD2; Genbank Accession No. NM_{—}004183 and No. NP_{—}004174; Swiss Prot. Accession Number_{—}076090; formerly Genbank Accession No. XM_{—}043405; see FIGS. 4A and 4B), or
(ii) a Bestrophin homologous protein on human chromosome 12 (genomic sequence Genbank Accession No. AC016153, identical to human VMD2-like protein 3, vitelliform macular dystrophy 2-like protein 3; Genbank Accession Number AF440758_{—}1, see FIGS. 4E and 4F),
(iii) a Bestrophin homologous protein on human chromosome 1 (genomic sequence Genbank Accession No. AL592166, identical to encoding a human VMD2-like protein 2, vitelliform macular dystrophy 2-like protein 2; Genbank Accession Number AF440757_{—}1, see FIGS. 4G and 4H), or
(iv) a Bestrophin homologous protein on human chromosome 19 (assembled from cDNA Genbank Accession No. NM_{—}017682 and genomic sequence AC018761, similar to human VMD2-like protein 1, vitelliform macular dystrophy 2-like protein 1 (Genbank Accession Number AF440756_{—}1) which has an additional 43 amino acids at the amino terminus, see FIGS. 4C and 4D or FIGS. 41 and 4J).

The invention particularly relates to a nucleic acid molecule encoding a polypeptide contributing to regulating the energy homeostasis and the metabolism of triglycerides or a portion thereof, wherein said nucleic acid molecule comprises
(a) a Bestrophin nucleotide sequence as shown in FIG. 4 or the complementary strand thereof,
(b) a nucleotide sequence which hybridizes under stringent conditions to a nucleic acid molecule encoding a Bestrophin amino acid sequence as shown in FIG. 4 or the complementary strand thereof,
(c) a nucleotide sequence corresponding to the sequences of (a) or (b) within the degeneration of the genetic code, (d) a nucleotide sequence which encodes a polypeptide which is at least 85%, preferably at least 90%, more preferably at least 95%, more preferably at least 98% and up to 99.6% identical to an amino acid sequence as shown in FIG. 4,
(e) a nucleotide sequence which differs from the nucleic acid molecule of (a) to (d) by mutation and wherein said mutation causes an alteration, deletion, duplication or premature stop in the encoded polypeptide, or
(f) a partial nucleotide sequence of any of the sequences of (a) to (e) having a length of at least 15 bases, preferably at least 20 bases, more preferably at least 25 bases and most preferably at least 50 bases.

The invention is based on the result that Bestrophin homologous proteins (for example, VMD2, VMD2-like protein 1, VMD2-like protein 2, or VMD2-like protein 3; herein also referred to as Bestrophin or Bestrophins) and the polynucleotides encoding these, are involved in the regulation of triglyceride storage and therefore energy homeostasis. Mostly preferred are nucleic acids encoding VMD2 or VMD2-like protein 3 and the corresponding proteins and effectors thereof.

The invention describes the use of these compositions for the diagnosis, study, prevention, or treatment of diseases and disorders related thereto, including metabolic diseases such as obesity as well as related disorders such as eating disorder, cachexia, diabetes mellitus, hypertension, coronary heart disease, hypercholesterolemia, osteoarthritis, gallstones, cancers of the reproductive organs, and sleep apnea.

To find genes with novel functions in energy homeostasis, metabolism, and obesity, a functional genetic screen was performed with the model organism Drosophila melanogaster (Meigen). One resource for screening was a Drosophila melanogaster stock collection of EP-lines. The P-vector of this collection has Gal4-UAS-binding sites fused to a basal promoter that can transcribe adjacent genomic Drosophila sequences upon binding of Gal4 to UAS-sites. This enables the EP-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

Triglycerides are the most efficient storage for energy in cells. Obese people mainly show an significant increase in the content of triglycerides. In order to isolate genes with a function in energy homeostasis, several thousand EP-lines were tested for their triglyceride content after a prolonged feeding period. Lines with significantly changed triglyceride content were selected as positive candidates for further analysis as, for example, but not for limiting the scope of the invention, is described below in the examples section.

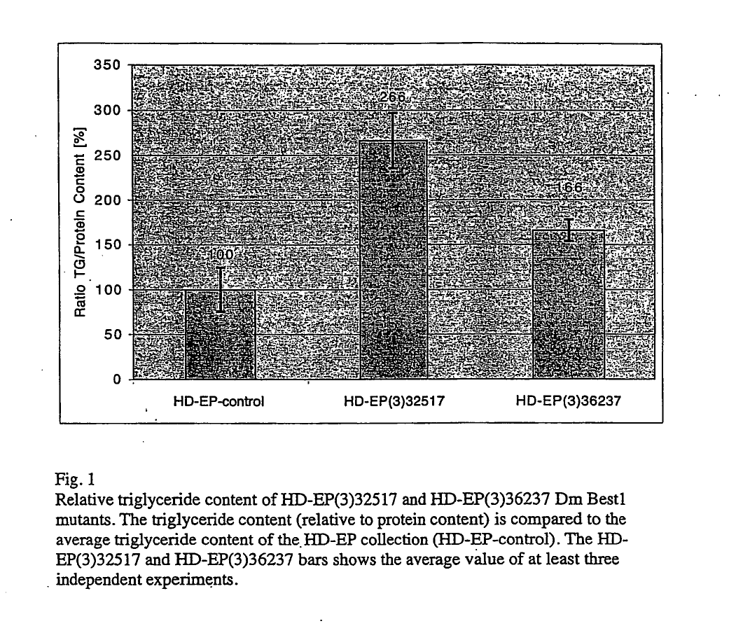

The result of the triglyceride content analysis is shown in FIG. 1. We found that homozygous HD-EP(3)32517 and HD-EP(3)36237 flies have a higher triglyceride content than the controls (average triglyceride levels). Therefore, the very likely loss of a gene activity in the gene locus 85F13-85F14 (estimated chromosomal localisation where the EP-vector of HD-EP(3)32517 and HD-EP(3)36237 flies is integrated) is responsible for changes in the metabolism of the energy storage triglycerides, therefore representing in both cases an obese fly model.

The increase of triglyceride content due to the loss of a gene function suggests gene activities in energy homeostasis in a dose dependent manner that controls the amount of energy stored as triglycerides.

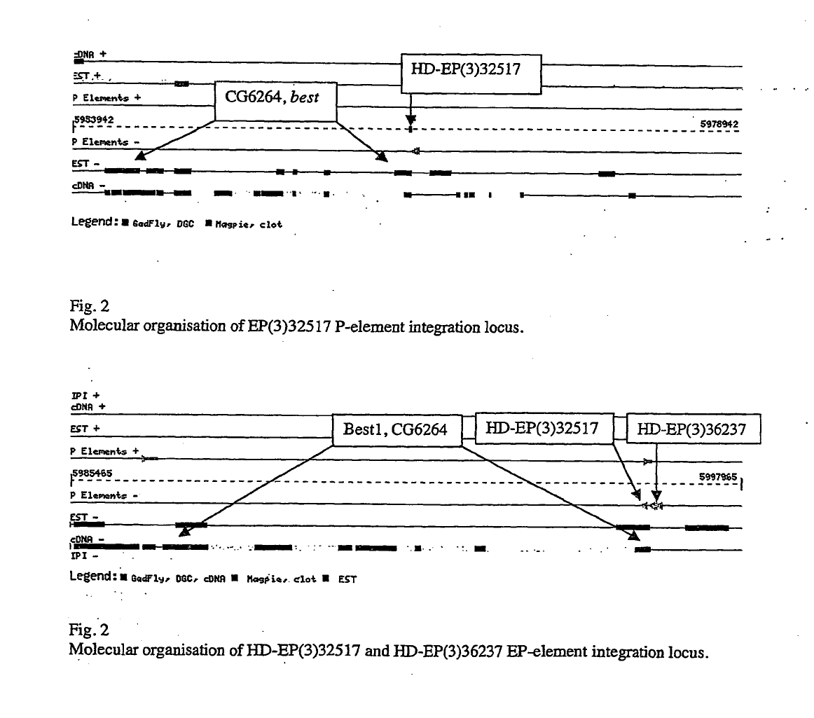

Nucleic acids encoding the Bestrophin protein of the present invention were identified using a plasmid-rescue technique. Genomic DNA sequences were isolated that are localised directly 5′ or 3′ to the EP(3)32517 and HD-EP(3)36237 integrations. Using those isolated genomic sequences public databases like Berkeley Drosophila Genome Project (GadFly) were screened thereby confirming the integration side of EP(3)32517 and HD-EP(3)36237 within a 5′ exon or enhancer/promoter region of the Bestrophin homologous gene (FIG. 2). FIG. 2 shows the molecular organisation of this locus. Genomic DNA sequence is represented by the assembly as a black dotted line in the middle that includes the integration site of EP(3)32517 and HD-EP(3)36237. Numbers represent the coordinates of the genomic DNA. Grey bars on the two cDNA-lines represent the predicted genes (GadFly & Magpie), and grey symbols on the P-Elements-line the EP-vector integration sites. Predicted exons of gene CG6264 are shown as dark grey bars and predicted introns as light grey bars.

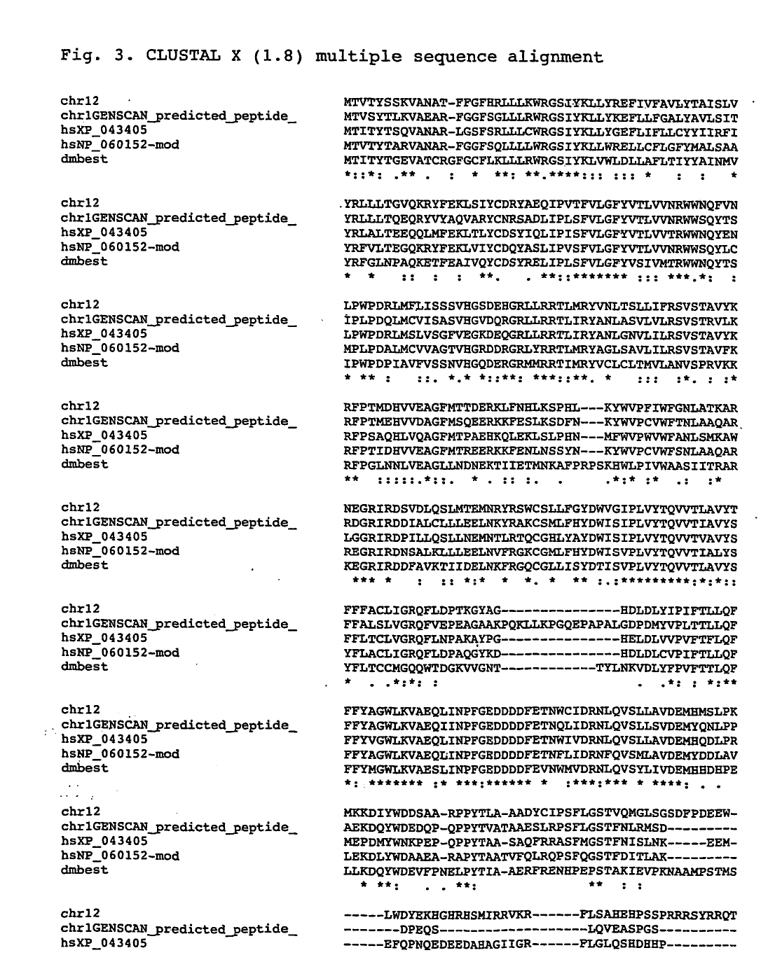

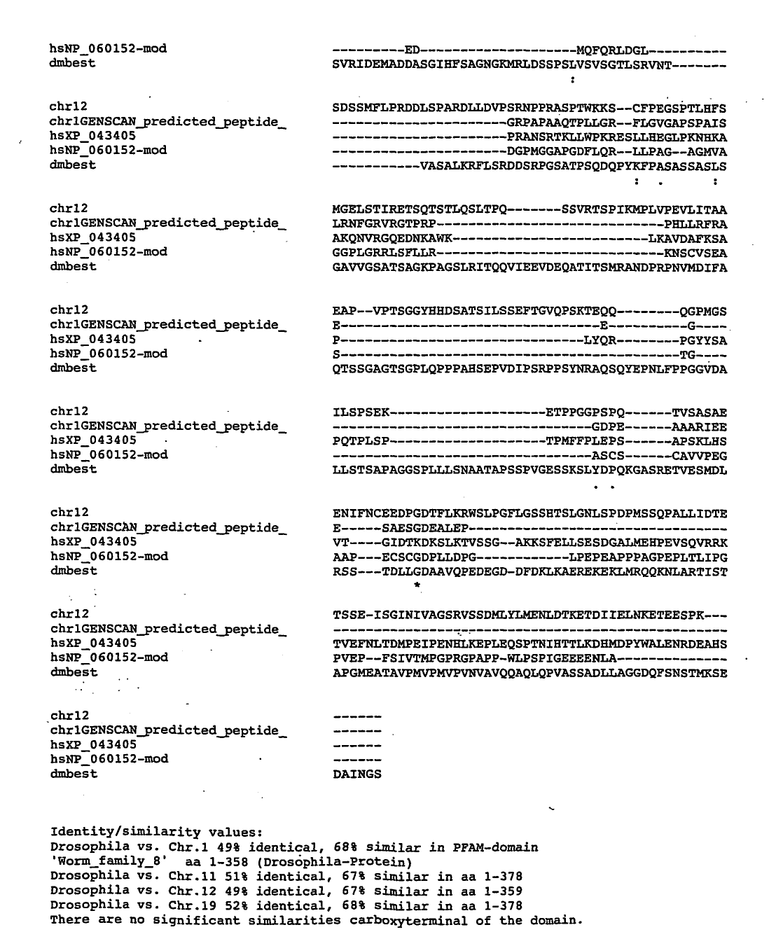

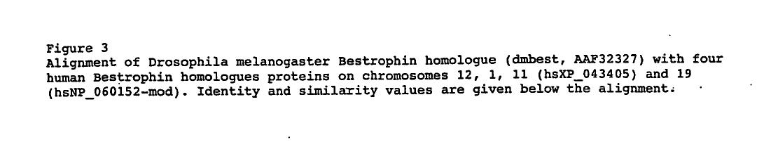

Bestrophin encodes for a gene that is predicted by GadFly sequence analysis programs (GadFly Accession Number CG6264). No functional data described the regulation of obesity and metabolic diseases are available in the prior art for the genes and proteins shown in FIGS. 3, 4, and 5, referred to as Bestrophin or Bestrophin homologues in the present invention.

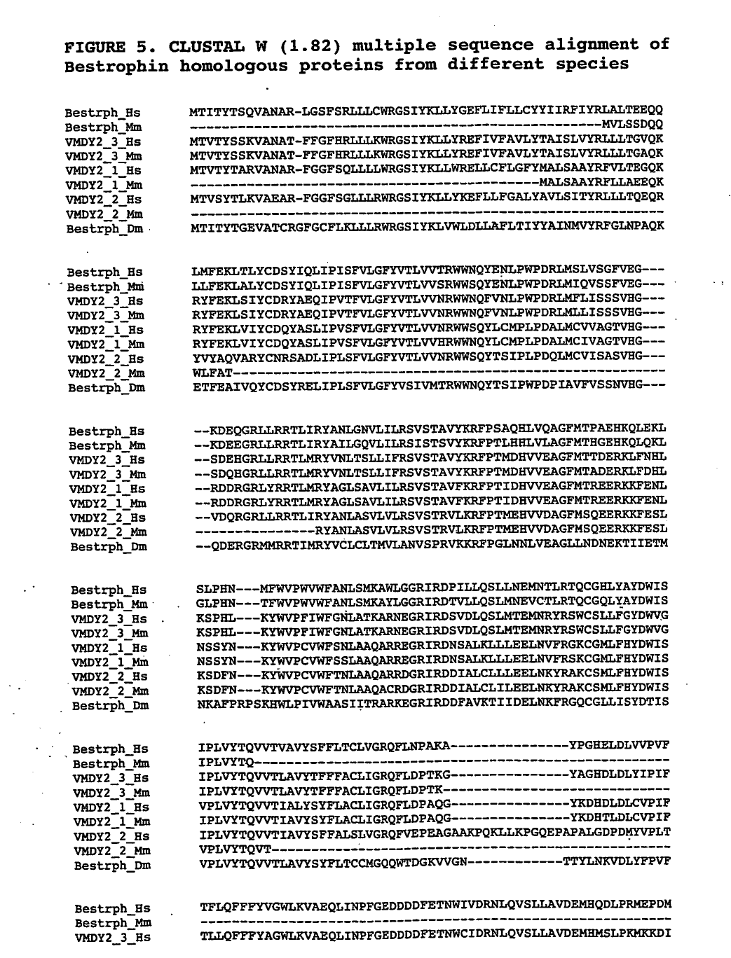

The present invention further relates to a polypeptide encoded by the nucleic acid as described above. Preferably the polypeptide comprises the amino acid sequence of Bestrophin. A comparison (Clustal X 1.8 or ClustaW 1.82) between the Bestrophin proteins of different species (human, mouse, and Drosophila) was conducted and is shown in FIGS. 3 and 5.

Using TMHMM protein analysis tools (A. Krogh, et al., Journal of Molecular Biology, 305(3):567-580, 2001.), it was found, for example, that the Bestrophin protein of the invention has at least four characteristic protein motifs (for example, transmembrane domains). These motifs are found throughout the whole Bestrophin famliy. InterPro analysis (Apweiler et al., Nucleic Acids Research 29:37-40, 2001) of the Drosophila gene (CG6264) indicated the presence of a worm-family-8 domain (Sonnhammer and Durbin, Genomics 46:200-216, 1997) typical for all Bestrophin homologous proteins. Based upon homology, Bestrophin proteins of the invention and each homologous protein or peptide may share at least some activity.

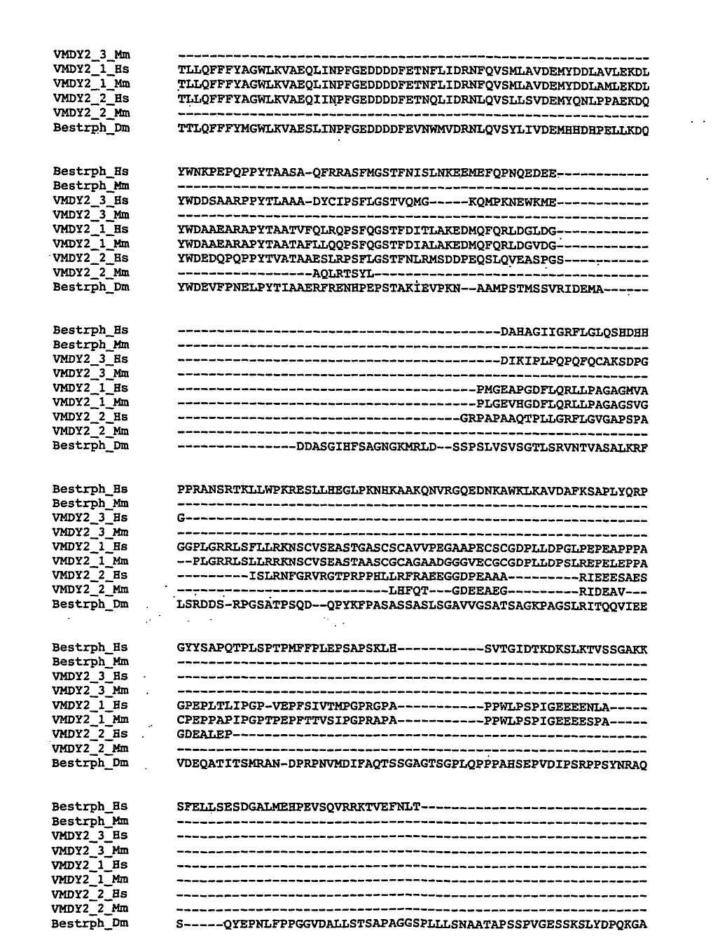

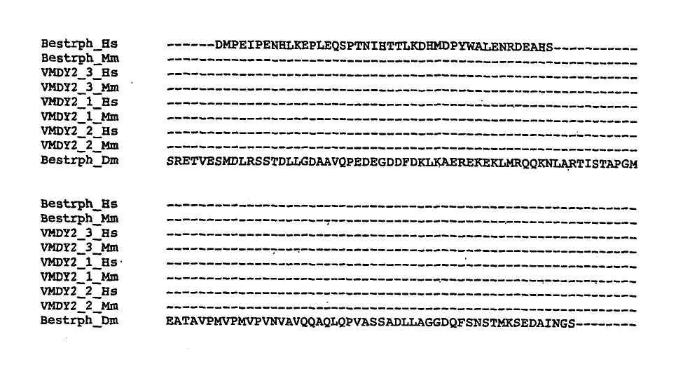

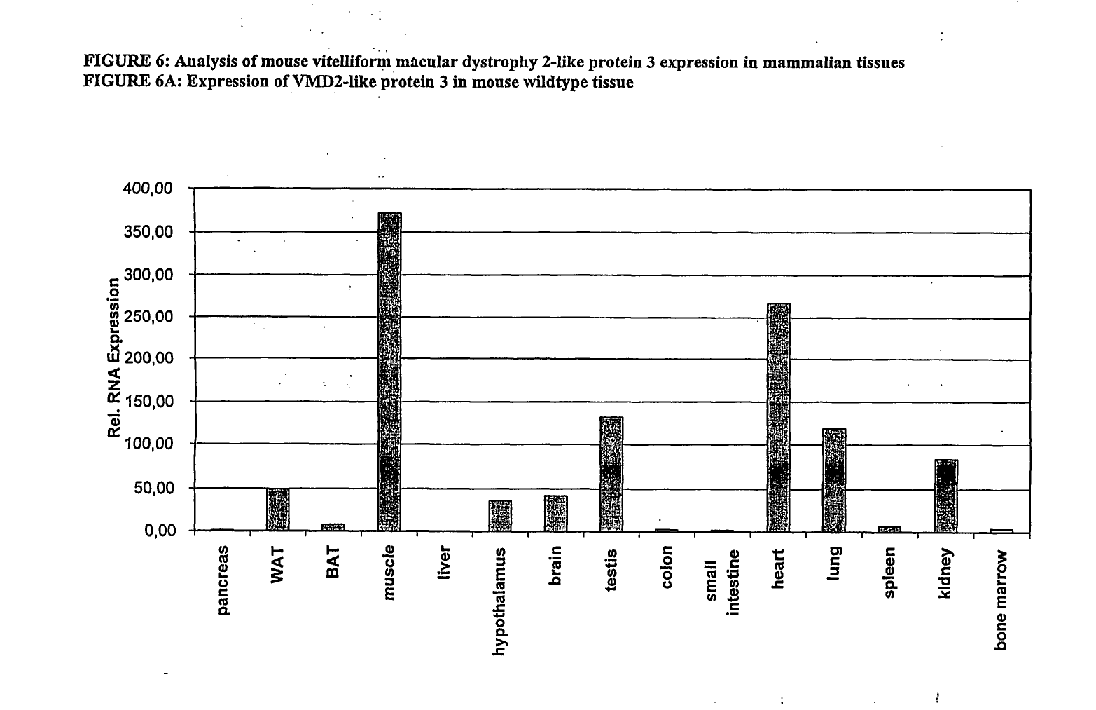

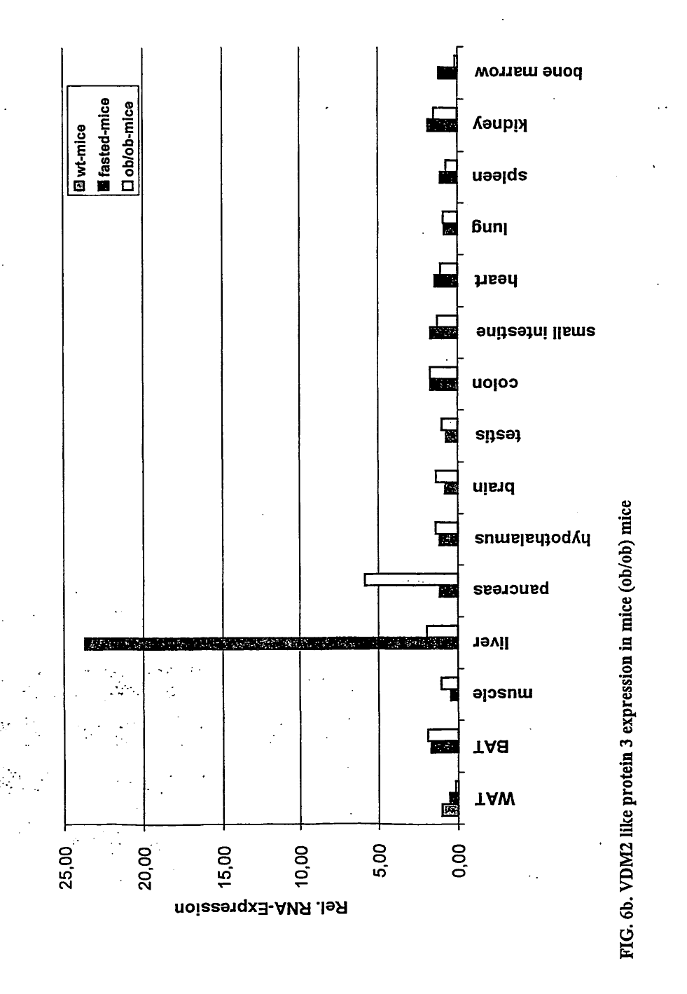

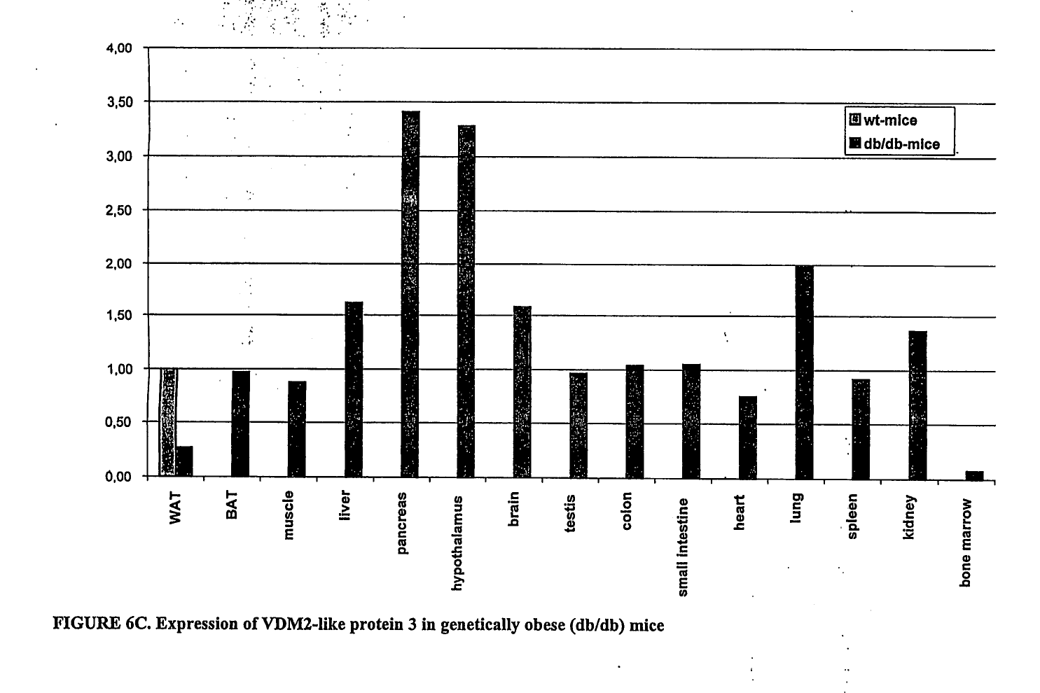

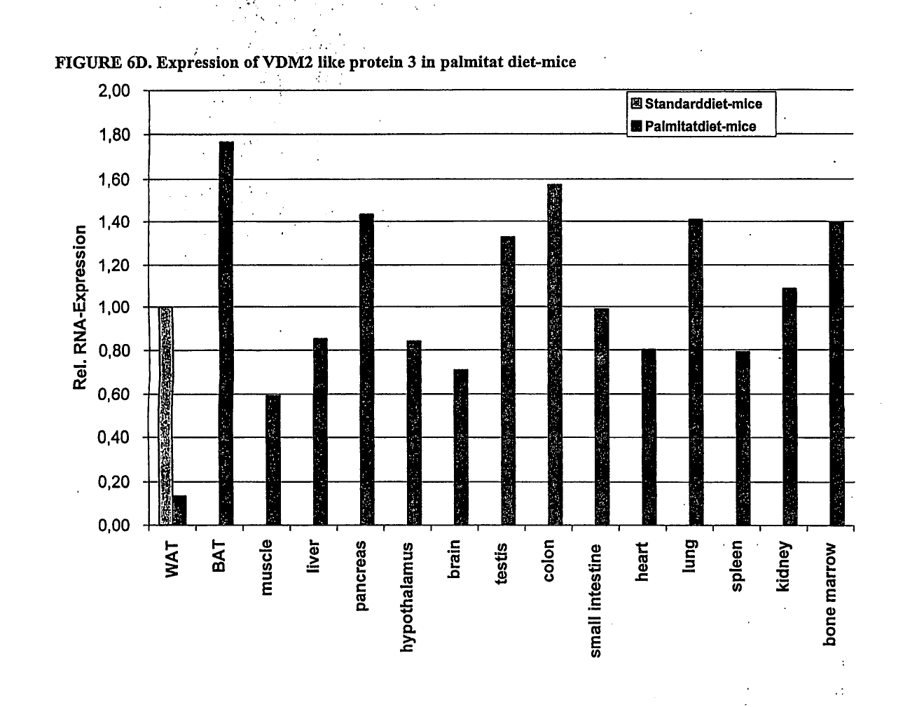

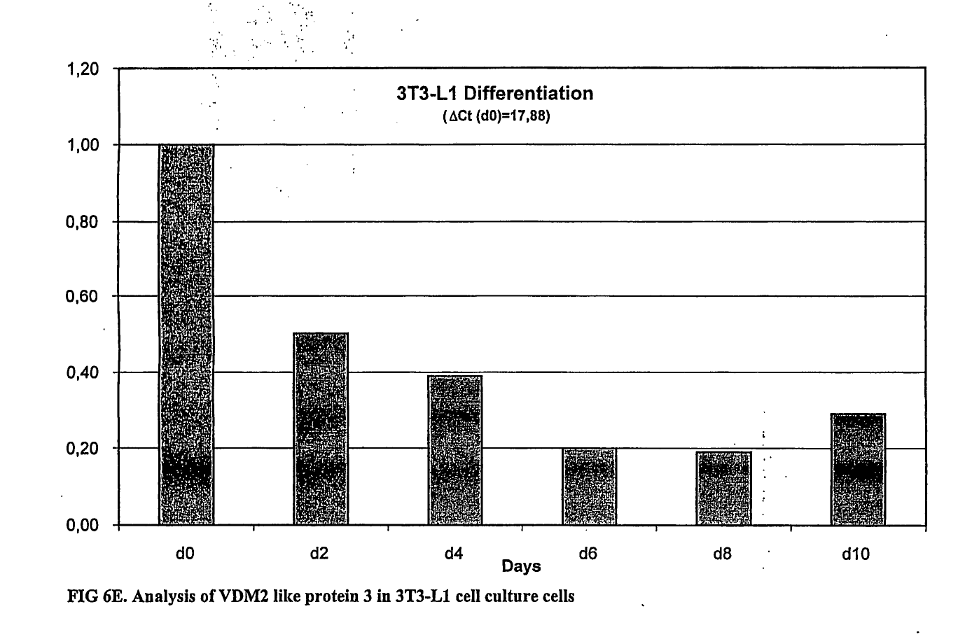

As shown in FIG. 6, VDM2-like protein 3 shows clear expression in adipose tissues. Under high fat diet, VDM2-like protein 3 is down-regulated in adipose tissues (WAT), suggesting that the protein is regulating the adipogenesis, possibly as inhibitor of this process. The expression of three Bestrophiris (VMD2, VMD2-like protein 1, VMD2-like protein 3) was observed in adipose tissues as well as in the hypothalamus and to a lesser degree in other brain areas. Thus, Bestrophins show a clear tissue specific expression suggesting distinct roles in the metabolism (see Examples 4 and 5 and FIGS. 6, 7 and 8 and FIGS. 9 and 10 respectively).

The invention also encompasses polynucleotides that encode Bestrophin and homologous proteins. Accordingly, any nucleic acid sequence, which encodes the amino acid sequences of Bestrophin, can be used to generate recombinant molecules that express Bestrophin. In a particular embodiment, the invention encompasses polynucleotides selected from human Bestrophin nucleic acids as described above or fragments or variants thereof. It will be appreciated by those skilled in the art that as a result of the degeneracy of the genetic code, a multitude of nucleotide sequences encoding Bestrophins, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequences of naturally occurring Bestrophins, and all such variations are to be considered as being specifically disclosed. Although nucleotide sequences which encode Bestrophins and their variants are preferably capable of hybridising to the naturally occurring nucleotide sequences of Bestrophins (for example the sequences encoding VDM2, VDM2-like protein 1, VDM2-like protein 2, or VDM2-like protein 3) under appropriately selected conditions of stringency, it may be advantageous to produce nucleotide sequences or their derivatives possessing a substantially different codon usage. Codons may be selected to increase the rate at which expression of the peptide occurs in a particular prokaryotic or eukaryotic host in accordance with the frequency with which particular codons are utilised by the host. Other reasons for substantially altering the nucleotide sequence encoding Bestrophins and its derivatives without altering the encoded amino acid sequences include the production of RNA transcripts having more desirable properties, such as a greater half-life, than transcripts produced from the naturally occurring sequences. The invention also encompasses production of DNA sequences or portions thereof and its derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding Bestrophin or any portion thereof.

Also encompassed by the invention are polynucleotide sequences that are capable of hybridizing to the claimed nucleotide sequences, under various conditions of stringency. Hybridization conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding Bestrophin which are encompassed by the invention include deletions, insertions, or substitutions of different nucleotides resulting in a polynucleotide that encodes the same or a functionally equivalent Bestrophin.

The encoded proteins may also contain deletions, insertions, or substitutions of amino acid residues, which produce a silent change and result in a functionally equivalent Bestrophin (for example, VDM2, VDM2-like protein 1, VDM2-like protein 2, or VDM2-like protein 3). Deliberate amino acid substitutions may be made on the basis of similarity in polarity, charge, solubility, hydrophobicity, hydrophilicity, and/or the amphipathic nature of the residues as long as the biological activity of Bestrophin is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoding Bestrophins (for example, VDM2, VDM2-like protein 1, VDM2-like protein 2, or VDM2-like protein 3). As used herein, an “allele” or “allelic sequence” is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence. These changes may be determined by DNA sequencing methods which are well known and generally available in the art.

The nucleic acid sequences encoding Bestrophin (for example, VDM2, VDM2-like protein 1, VDM2-like protein 2, or VDM2-like protein 3) may be extended utilising a partial nucleotide sequence and employing various methods known in the art to detect upstream sequences such as promoters and/or regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences, which contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions. Capillary electrophoresis systems, which are commercially available, may be used to analyse the size or confirm the nucleotide sequence of sequencing or PCR products.

In another embodiment of the invention, polynucleotide sequences or fragments thereof which encode Bestrophin, or fusion proteins or functional equivalents thereof, may be used in recombinant DNA molecules to direct expression of Bestrophin in appropriate host cells. Due to the inherent degeneracy of the genetic code, other DNA sequences, which encode substantially the same, or a functionally equivalent amino acid sequence may be produced and these sequences may be used to clone and express Bestrophin. As will be understood by those of skill in the art, it may be advantageous to produce Bestrophin encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. The nucleotide sequences of the present invention can be engineered using methods generally known in the art in order to alter Bestrophin encoding sequences for a variety of reasons, including but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences encoding Bestrophin (for example, VDM2, VDM2-like protein 1, VDM2-like protein 2, or VDM2-like protein 3) may be ligated to a heterologous sequence to encode a fusion protein. For example, to screen peptide libraries for modulators, e.g. inhibitors of Bestrophin activities, it may be useful to use chimeric Bestrophin proteins that can be recognised by a commercially available antibodies. A fusion protein may also be engineered to contain a cleavage site located between the Bestrophin encoding sequence and the heterologous protein sequences, so that Bestrophin may be cleaved and purified away from the heterologous moiety. In another embodiment, sequences encoding Bestrophin may be synthesised, in whole or in part, using chemical methods well known in the art (see Caruthers, M. H. et al. (1980) Nucl. Acids Res. Symp. Ser. 7:215-223, Horn, T. et al. (1980) Nucl. Acids Res. Symp. Ser. 7:225-232). Alternatively, the proteins themselves may be produced using chemical methods to synthesise the amino acid sequence of Bestrophin, or a portion thereof. For example, peptide synthesis can be performed using various solid-phase techniques (Roberge, J. Y. et al. (1995) Science 269:202-204) and automated synthesis may be achieved, for example, using the ABI 431A peptide synthesiser (Perkin Elmer). The newly synthesised peptide may be substantially purified by preparative high performance liquid chromatography (e.g., Creighton, T. (1983) Proteins, Structures and Molecular Principles, WH Freeman and Co., New York, N.Y.). The composition of the synthetic peptides may be confirmed by amino acid analysis or sequencing (e.g., the Edman degradation procedure; Creighton, supra). Additionally, the amino acid sequences of Bestrophin, or any part thereof, may be altered during direct synthesis and/or combined using chemical methods with sequences from other proteins, or any part thereof, to produce a variant polypeptide.

In order to express a biologically active Bestrophin (for example, VDM2, VDM2-like protein 1, VDM2-like protein 2, or VDM2-like protein 3), the nucleotide sequences encoding Bestrophin functional equivalents, may be inserted into appropriate expression vectors, i.e., a vector, which contains the necessary elements for the transcription and translation of the inserted coding sequence. Methods, which are well known to those skilled in the art, may be used to construct expression vectors containing sequences encoding Bestrophin and appropriate transcriptional and translational control elements. These methods include in vitro recombinant DNA techniques synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilised to contain and express sequences encoding Bestrophin (for example, VDM2, VDM2-like protein 1, VDM2-like protein 2, or VDM2-like protein 3). These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems. The vectors may comprise “control elements” or “regulatory sequences”, i.e. non-translated regions including enhancers, promoters, 5′ and 3′ untranslated regions which interact with host cellular proteins to carry out transcription and translation. Such elements may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Gibco BRL) and the like may be used. The baculovirus polyhedrin promoter may be used in insect cells. Promoters and enhancers derived from the genomes of plant cells (e.g., heat shock, RUBISCO; and storage protein genes) or from plant viruses (e.g., viral promoters and leader sequences) may be cloned into the vector. In mammalian cell systems, promoters from mammalian genes or from mammalian viruses are preferable. If it is necessary to generate a cell line that contains multiple copies of the sequences encoding Bestrophin, vectors based on SV40 or EBV may be used with an appropriate selectable marker.

In bacterial systems, a number of expression vectors may be selected depending upon the use intended for Bestrophin. For example, when large quantities of Bestrophin are needed for the induction of antibodies, vectors, which direct high level expression of fusion proteins that are readily purified, may be used. Such vectors include, but are not limited to, the multifunctional E. coli cloning and expression vectors such as the BLUESCRIPT phagemid (Stratagene), pIN vectors (Van Heeke, G. and S. M. Schuster (1989) J. Biol. Chem. 264:5503-5509); and the like. PGEX vectors (Promega, Madison, Wis.) may also be used to express foreign polypeptides as fusion proteins with Glutathione S-Transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will. In the yeast, Saccharomyces cerevisiae, a number of vectors containing constitutive or inducible promoters such as alpha factor, alcohol oxidase, and PGH may be used. For reviews, see Ausubel et al., (supra) and Grantet al. (1987) Methods Enzymol. 153:5.16-544.

In cases where plant expression vectors are used, the expression of sequences encoding Bestrophin may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express Bestrophin. For example, in one such system, Autographa californica nuclear polyhedrosis virus (AcNPV) is used as a vector to express foreign genes in Spodoptera frugiperda cells or in Trichoplusia larvae (Engelhard, E. K. et al. (1994) Proc. Nat. Acad. Sci. 91:3224-3227).

In mammalian host cells, a number of non-viral or viral expression systems may be utilised. In cases where an adenovirus is used as an expression vector, sequences encoding Bestrophin may be ligated into an adenovirus transcription/translation complex consisting of the late promoter and tripartite leader sequence. Insertion in a non-essential E1 or E3 region of the viral genome may be used to obtain viable viruses which are capable of expressing Bestrophin in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is preferred. For example, cell lines which stably express Bestrophin may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells, which successfully express the introduced sequences. Resistant clones of stably transformed cells may be proliferated using tissue culture techniques appropriate to the cell type. Any number of selection systems may be used to recover transformed cell lines. These include, but are not limited to, the herpes simplex virus thymidine kinase (Wigler, M. et al. (19.77) Cell 11:223-32) and adenine phosphoribosyltransferase (Lowy, I. et al. (1980) Cell 22:817-23) genes, which can be employed in tk⁻ or aprt⁻ cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilise indole in place of tryptophan, or hisD, which allows cells to utilise histinol in place of histidine (Hartman, S. C. and R. C. Mulligan (1988) Proc. Natl. Acad. Sci. 85:8047-51). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequences encoding Bestrophin are inserted within a marker gene sequence, recombinant cells containing sequences encoding Bestrophin can be identified by the absence of marker gene function. Alternatively, a marker gene can be placed in tandem with sequences encoding Bestrophin under the control of a single promoter. Expression of the marker gene in response to induction or selection usually indicates expression of the tandem gene as well. Alternatively, host cells, which contain the nucleic acid sequences encoding Bestrophin and express Bestrophin, may be identified by a variety of procedures known to those of skill in the art. These procedures include, but are not limited to, DNA-DNA, or DNA-RNA hybridisation and protein bioassay or immunoassay techniques which include membrane, solution, or chip based technologies for the detection and/or quantification of nucleic acid or protein.

The presence of polynucleotide sequences encoding Bestrophin can be detected by DNA-DNA or DNA-RNA hybridisation or amplification using primers, probes or portions or fragments of polynucleotides encoding Bestrophin. Nucleic acid amplification based assays involve the use of oligonucleotides or oligomers based on the sequences encoding Bestrophin to detect transformants containing DNA or RNA encoding Bestrophin. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of Bestrophin, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilising monoclonal antibodies reactive to two non-interfering epitopes on Bestrophin is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labelled hybridisation or PCR probes for detecting sequences related to polynucleotides encoding Bestrophin include oligo-labelling, nick translation, end-labelling or PCR amplification using a labelled nucleotide.

Alternatively, the sequences encoding Bestrophin (for example, VDM2, VDM2-like protein 1, VDM2-like protein 2, or VDM2-like protein 3), or any portion thereof may be cloned into a vector for the production of an mRNA probe. Such vectors are known in the art, are commercially available, and may be used to synthesise RNA probes in vitro by addition of an appropriate RNA polymerase such as T7, T3, or SP6 and labelled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding Bestrophin may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode Bestrophin may be designed to contain signal sequences, which direct secretion of Bestrophin through a prokaryotic or eukaryotic cell membrane. Other recombinant constructs may be used to join sequences encoding Bestrophin to nucleotide sequence encoding a polypeptide domain, which will facilitate purification of soluble proteins. Such purification facilitating domains include, but are not limited to, metal chelating peptides such as histidine-tryptophan modules that allow purification on immobilised metals, protein A domains that allow purification on immobilised immunoglobulin, and the domain utilised in the FLAG extension affinity purification system (Immunex Corp., Seattle, Wash.) The inclusion of cleavable linker sequences such as those specific for Factor XA or Enterokinase (Invitrogen, San Diego, Calif.) between the purification domain and Bestrophin may be used to facilitate purification. One such expression vector provides for expression of a fusion protein containing Bestrophin and a nucleic acid encoding 6 histidine residues preceding a Thioredoxine or an Enterokinase cleavage site. The histidine residues facilitate purification on IMIAC (immobilised metal ion affinity chromatography as described in Porath, J. et al. (1992, Prot. Exp. Purif. 3: 263-281)) while the Enterokinase cleavage site provides a means for purifying Bestrophin from the fusion protein. A discussion of vectors which contain fusion proteins is provided in Kroll, D. J. et al. (1993; DNA Cell Biol. 12:441-453). In addition to recombinant production, fragments of Bestrophin may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesiser (Perkin Elmer). Various fragments of Bestrophin may be chemically synthesised separately and combined using chemical methods to produce the full length molecule. The data disclosed in this invention show that the nucleic acids and proteins of the invention and effectors thereof are useful in diagnostic and therapeutic applications implicated, for example but not limited to, in metabolic disorders such as obesity as well as related disorders such as eating disorder, cachexia, diabetes mellitus, hypertension, coronary heart disease, hypercholesterolemia, osteoarthritis, gallstones, cancers of the reproductive organs, and sleep apnea. Hence, diagnostic and therapeutic uses for the Bestrophin nucleic acids and proteins are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are particularly useful in diagnostic and therapeutic applications as described below. For example, but not limited to, cDNAs encoding the Bestrophin proteins of the invention and particularly their human homologues may be useful in gene therapy, and the Bestrophin proteins of the invention and particularly their human homologues may be useful when administered to a subject in need thereof. By way of non-limiting example, the compositions of the present invention will have efficacy for prevention or treatment of patients suffering from diseases and disorders as described above.

Bestrophin nucleic acids, or fragments thereof, may further be useful in diagnostic applications, wherein the presence or amount of the nucleic acids or the proteins are to be assessed. Bestrophin proteins or fragments thereof are further useful in the generation of antibodies that bind immunospecifically to the protein of the invention for use in therapeutic or diagnostic methods.

For example, in one aspect, antibodies which are specific for Bestrophin may be used directly as an antagonist, or indirectly as a targeting or delivery mechanism for bringing a pharmaceutical agent to cells or tissue which express Bestrophin. The antibodies may be generated using methods that are well known in the art. Such antibodies may include, but are not limited to, polyclonal, monoclonal, chimeric, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunised by injection with Bestrophin any fragment or oligopeptide thereof which has immunogenic properties. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable. It is preferred that the peptides, fragments, or oligopeptides used to induce antibodies to Bestrophin have an amino acid sequence consisting of at least five amino acids, and more preferably at least 10 amino acids. It is preferable that they are identical to a portion of the amino acid sequence of the natural protein, and they may contain the entire amino acid sequence of a small, naturally occurring molecule. Short stretches of Bestrophin amino acids may be fused with those of a heterologous protein such as keyhole limpet hemocyanin and antibodies produced against the chimeric molecule.

Monoclonal antibodies to Bestrophin may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used. (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce Bestrophin-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Fragments of anti-Bestrophin antibodies, which contain specific binding sites for Bestrophin, may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between Bestrophins and their specific-antibody.

In another embodiment of the invention, the polynucleotides encoding Bestrophin, or any fragment thereof, or effector nucleic acids such as aptamers, antisense molecules, ribozymes or RNAi molecules may be used for therapeutic purposes. In one aspect, aptamers, i.e. nucleic acid molecules capable of binding to a target protein and modulating its activity may be obtained by known methods, e.g. by affinity selection of combinatorial nucleic acid libraries.

In a further aspect, antisense molecules may be used in situations in which it would be desirable to block the transcription of the mRNA. In particular, cells may be transformed with sequences complementary to polynucleotides encoding Bestrophin. Thus, antisense molecules may be used to modulate Bestrophin activity, or to achieve regulation of gene function. Such technology is now well know in the art, and sense or antisense oligomers or larger fragments, can be designed from various locations along the coding and/or control regions of sequences encoding Bestrophin. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the gene encoding Bestrophin. These techniques are described both in Sambrook et al. (supra) and in Ausubel et al. (supra). Genes encoding Bestrophin can be turned off by transforming a cell or tissue with expression vectors which express high levels of polynucleotide or fragment thereof which encodes Bestrophin. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA, or nucleic acid analogues such as PNA, to the control regions of the gene encoding Bestrophin, i.e., the promoters, enhancers, and/or introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridisation of the ribozyme molecule to complementary target RNA, followed by endonucleolytic cleavage. Examples, which may be used, include engineered hammerhead motif ribozyme molecules that can be specifically and efficiently catalyse endonucleolytic cleavage of target sequences. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridisation with complementary oligonucleotides using ribonuclease protection assays.

Effector nucleic acids such as antisense molecules and ribozymes may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesise RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of Bestrophin, comprise as an active ingredient the protein, the nucleic acid coding therefor or a receptor recognizing the protein or the nucleic acid, e.g. antibodies to Bestrophin, mimetics, agonists, antagonists, activators or inhibitors of Bestrophin. The compositions may be administered alone or in combination with at least one other agent, such as stabilising compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping, or lyophilising processes. After pharmaceutical compositions have been prepared, they can be placed in an appropriate container and labelled for treatment of an indicated condition. For administration of Bestrophin, such labelling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example Bestrophin, fragments thereof, antibodies of Bestrophin, which is effective against a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of is delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies may be used for the diagnosis of conditions or diseases characterised by or associated with over- or underexpression of Bestrophin, or in assays to monitor patients being treated with Bestrophin, agonists, antagonists or inhibitors. The antibodies useful for diagnostic purposes may be prepared in the same manner as those described above for therapeutics. Diagnostic assays for Bestrophin include methods, which utilise the antibody and a label to detect Bestrophin in human body fluids or extracts of cells or tissues. The antibodies may be used with or without modification, and may be labelled by joining them, either covalently or non-covalently, with a reporter molecule. A wide variety of reporter molecules which are known in the art may be used several of which are described above.

A variety of protocols including ELISA, RIA, and FACS for measuring Bestrophin are known in the art and provide a basis for diagnosing altered or abnormal levels of Bestrophin expression. Normal or standard values for Bestrophin expression may be established by combining body fluids or cell extracts taken from normal mammalian subjects, preferably human subjects, with antibodies to Bestrophin under conditions suitable for complex formation. The amount of standard complex formation may be quantified by various methods, but preferably by photometry, means. Quantities of Bestrophin expressed in control and disease samples e.g. from biopsied tissues may be compared with the standard values. Deviation between standard and subject values establishes the parameters for diagnosing disease.

In another embodiment of the invention, the polynucleotides specific for Bestrophin may be used for diagnostic purposes. The polynucleotides, which may be used, include oligonucleotide sequences, antisense RNA and DNA molecules, and nucleic acid analogues such as PNAs. The polynucleotides may be used to detect and quantitate gene expression in samples, e.g. in biopsied tissues in which expression is correlated with disease. The diagnostic assay may be used to distinguish between absence, presence, and excess protein expression, and/or to monitor regulation of Bestrophin levels during therapeutic intervention.

In one aspect, hybridisation with probes and/or primers which are capable of detecting polynucleotide sequences, including genomic sequences, encoding Bestrophin closely related molecules, may be used to identify nucleic acid sequences which encode Bestrophin. The specificity of the probe, whether it is made from a highly specific region, e.g., unique nucleotides in the 5′ regulatory region, or a less specific region, e.g., especially in the 3′ coding region, and the stringency of the hybridisation or amplification (maximal, high, intermediate, or low) will determine whether the probe identifies only naturally occurring sequences encoding Bestrophin or alleles, or related sequences. Probes may also be used for the detection of related sequences, and should preferably contain at least 50% of the nucleotides from any of the Bestrophin encoding sequences. The hybridisation probes of the subject invention may be DNA, RNA or nucleic acid analogues which are preferably derived from the nucleotide sequence of human Bestrophin cDNAs or RNAs, or from a genomic sequence including promoter, enhancer elements, and/or introns of the naturally occurring sequence. Means for producing specific hybridisation probes for DNAs encoding Bestrophin include the cloning of nucleic acid sequences encoding Bestrophin derivatives into vectors for the production of mRNA probes. Such vectors are known in the art, commercially available, and may be used to synthesise RNA probes in vitro by means of the addition of the appropriate RNA polymerases and the appropriate labelled nucleotides. Hybridisation probes may be labelled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences specific for Bestrophin (for example, VDM2, VDM2-like protein 1, VDM2-like protein 2, or VDM2-like protein 3) may be used for the diagnosis of conditions or diseases, which are associated with expression of Bestrophin. Examples of such conditions or diseases include, but are not limited to, metabolic diseases and disorders, such as obesity and diabetes. Polynucleotide sequences may also be used to monitor the progress of patients receiving treatment for metabolic diseases and disorders, including obesity and diabetes. The polynucleotide sequences encoding Bestrophin may be used in Southern or Northern analysis, dot blot, or other membrane-based technologies; in PCR technologies; or in dip stick, pin, ELISA or chip assays utilising fluids or tissues from patient biopsies to detect altered Bestrophin expression. Such qualitative or quantitative methods are well known in the art.

In a particular aspect, the nucleotide sequences may be useful in assays that detect activation or induction of various metabolic diseases such as obesity as well as related disorders such as eating disorder, cachexia, diabetes mellitus, hypertension, coronary heart disease, hyperchotesterolemia, osteoarthritis, gallstones, cancers of the reproductive organs, and sleep apnea. The nucleotide sequences may be labelled by standard methods, and added to a fluid or tissue sample from a patient under conditions suitable for the formation of hybridisation complexes. After a suitable incubation period, the sample is washed and the signal is quantitated and compared with a standard value. The presence of signal values corresponding to altered levels of nucleotide Bestrophin sequences in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of Bestrophin, a normal or standard profile for expression is established. This may be accomplished by combining body fluids or cell extracts taken from normal subjects, either animal or human subjects with a sequence, suitable as a probe or a primer, under conditions suitable for hybridisation or amplification. Standard hybridisation may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridisation assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as obesity as well as related disorders such as eating disorder, cachexia, diabetes mellitus, hypertension, coronary heart disease, hypercholesterolemia, osteoarthritis, gallstones, cancers of the reproductive organs, and sleep apnea the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the pancreatic diseases and disorders. Additional diagnostic uses for oligonucleotides may involve the use of PCR. Such oligomers may be chemically synthesised, generated enzymatically, or produced from a recombinant source. Oligomers will preferably consist of two nucleotide sequences, one with sense orientation (5′.fwdarw.3′) and another with antisense (3′.rarw.5′), employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of Bestrophin include radiolabelling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236). The speed of quantification of multiple samples may be accelerated by running the assay in an ELISA format where the oligomer of interest is presented in various dilutions and a spectrophotometric or calorimetric response gives rapid quantification.

In another embodiment of the invention, the nucleic acid sequences specific for Bestrophin may also be used to generate hybridisation probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes; bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding Bestrophin on a physical chromosomal map and a specific disease, or predisposition to a specific disease, may help to delimit the region of DNA associated with that genetic disease.

The nucleotide sequences of the subject invention may be used to detect differences in gene sequences between normal, carrier or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11 q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the proteins of the invention, its catalytic or immunogenic fragments or oligopeptides thereof, an in vitro model, a genetically altered cell or animal, can be used for screening libraries of compounds in any of a variety of drug screening techniques. One can identify effectors, e.g. receptors, enzymes, ligands or substrates that bind to, modulate or mimic the action of one or more of the proteins of the invention. The protein or fragment thereof employed in such screening may be free in solution, affixed to a solid support, borne on a cell surface, or located intracellularly. The formation of binding complexes, between the proteins of the invention and the agent tested, may be measured. Agents could also, either directly or indirectly, influence the activity of the proteins of the invention. Target mechanisms could for example include the chloride channel or other conductance activities of bestrophin as well as the regulation of bestrophin activity by phosphorylation and dephosphorylation or other posttranslational modifications. Moreover, agents could interfere with the dimerization or oligomerization of bestrophins or, in a heterologous manner, of bestrophins with other proteins, e.g. ion channels. Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to the proteins of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding the protein specifically compete with a test compound for binding the protein. In this manner, the antibodies can be used to detect the presence of any peptide, which shares one or more antigenic determinants with the protein.

The nucleic acids encoding the proteins of the invention can be used to generate transgenic cell lines and animals. These transgenic animals are useful in the study of the function and regulation of the proteins of the invention in vivo. Transgenic animals, particularly mammalian transgenic animals, can serve as a model system for the investigation of many developmental and cellular processes common to humans. Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a Bestrophin nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

The kit may be used for diagnostic or therapeutic purposes or for screening applications as described above. The kit may further contain user instructions.

The Figures show:

FIG. 1 shows the increase of triglyceride content of EP(3)32517 and HD-EP(3)36237 flies caused by homozygous viable integration of the P-vector (in comparison to controls).

FIG. 2 shows the molecular organisation of the mutated Bestrophin (Best1, CG6264) gene locus.

FIG. 3 shows the comparison (CLUSTAL X 1.8) of human Bestrophin proteins with the homologue Drosophila Bestrophin protein. Gaps in the alignment are represented as -. In the Figure, chr 12′ refers to human VDM2-like protein 3, chr1GENSCAN predicted_peptide′ refers to human VDM2-like protein 2, hsXP_{—}043405′ refers to human VDM2 protein, hs NP_{—}060152 mod′ refers to a protein similar to human VDM2-like protein 1, and dmbest′ refers to Drosophila melanogaster bestrophin.

FIG. 4 shows the nucleotide sequence of four human Bestrophin homologues (SEQ ID NO. 1-8).

FIG. 5 shows the comparison (CLUSTAL W 1.82 multiple sequence alignment) of human, mouse, and Drosophila Bestrophin proteins. Gaps in the alignment are represented as—
Bestrph_Hs refers to |SWISS-PROT Accession Number 076090 or GenBank Accession Number NP_{—}004174;
Bestrph_Mm refers to Mouse EnsEMBL Genscan predicted peptide 19.9000001-10000000.20.329852.331536;
VMDY2_{—}3_Hs GenBank Accession Number AF440758_{—}1, human vitelliform macular dystrophy 2-like protein 3;
VMDY2_3_Mm refers to ENSEMBL Accession Number ENSMUSP00000020378;
VMDY2_{—}1_Hs refers to GenBank Accession Number AF440756_{—}1, human vitelliform macular dystrophy 2-like protein 1;
VMDY2_{—}1 Mm refers to GenBank Accession Number NP_{—}663363 or ENSEMBL GenBank Accession Number ENSMUSP00000005276;
VMDY2_{—}2 Hs refers to GenBank Accession Number AF440757_{—}1, human vitelliform macular dystrophy 2-like protein 2 [Homo sapiens];
VMDY2_{—}2 Mm refers to ENSEMBL Accession Number ENSMUSP00000049289; and Bestrph_Dm′ refers to GenBank Accession Number NP_{—}652603.1.

FIG. 6 shows the analysis of vitelliform macular dystrophy 2-like protein 3 expression in mammalian tissues.

FIG. 6A. Real time PCR of VDM2-like protein 3 mRNA expression in mouse wildtype tissues.

FIG. 6B. VDM2-like protein 3 mRNA expression in mice-expressing leptin (wt mice) compared to genetically obese (ob/ob) mice without leptin expression and to fasted mice (fasted-mice).

FIG. 6C. Real-time PCR mediated analysis of vitelliform macular dystrophy 2 (VDM2)-like protein 3 mRNA in genetically obese (db/db) mice without leptin receptor expression compared to wild-type (wt) mice.

FIG. 6D. Expression of VDM2-like protein 3 mRNA in high fat (palmitat) diet-mice compared to mice fed a normal diet.

FIG. 6E. Expression of VDM-like protein 3 mRNA in mammalian fibroblast (3T3-L1) cells, during the differentiation from pre-adipocytes to mature adipocytes.

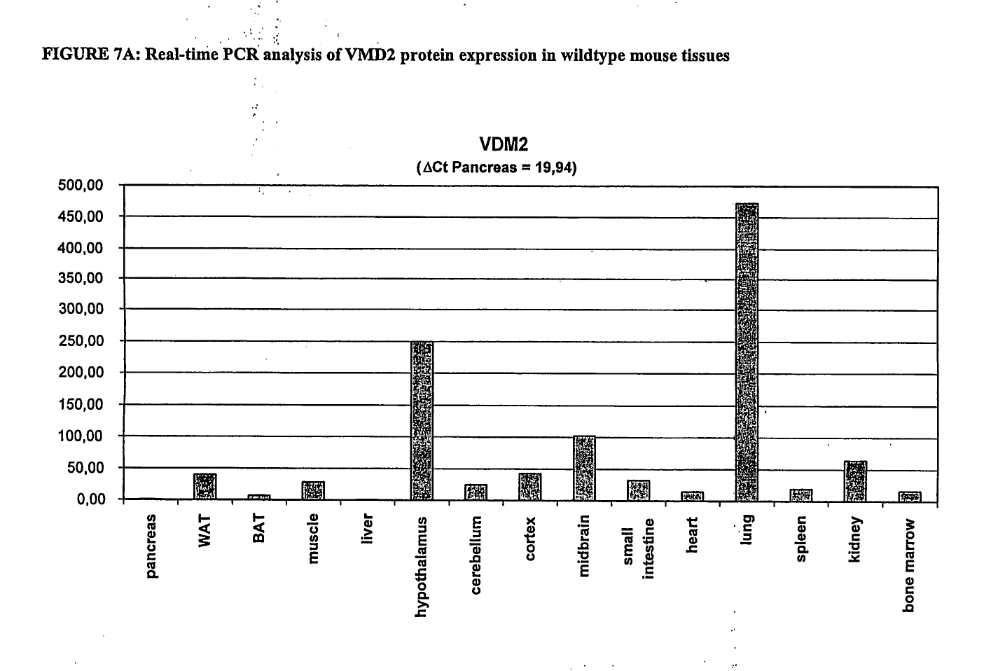

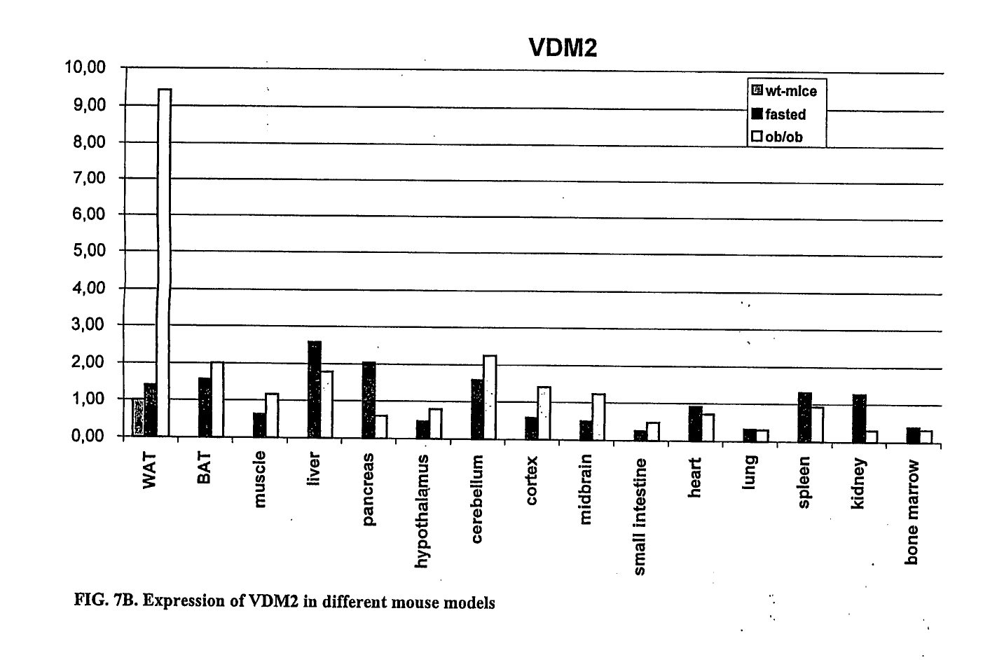

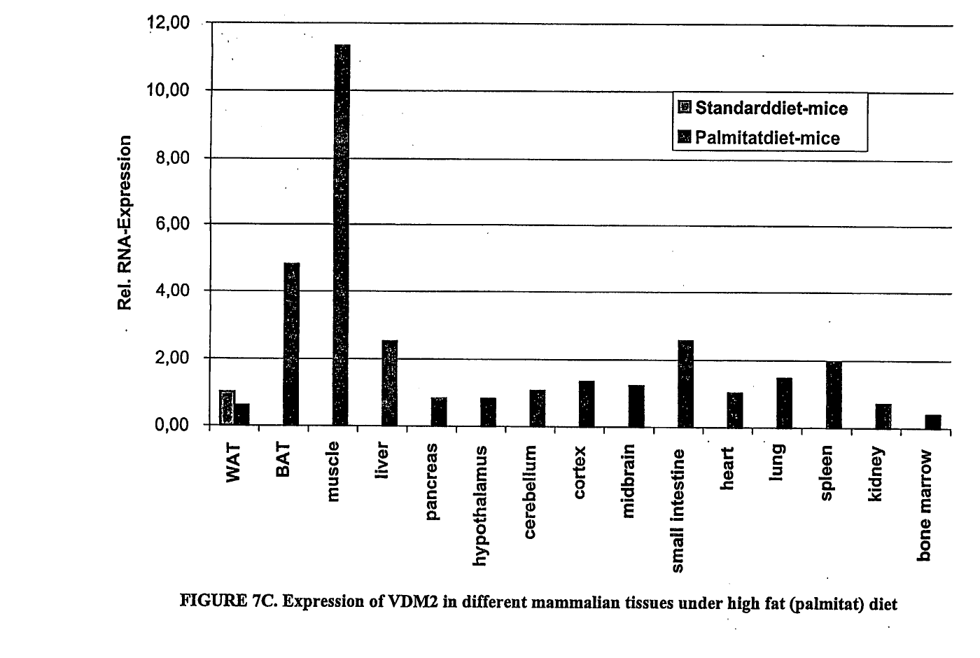

FIG. 7A shows the expression of mouse vitelliform macular dystrophy protein (VDM2) mRNA in different mouse tissues.

FIG. 7B shows the expression vitelliform macular dystrophy (VDM2) protein mRNA in different mouse models, (wildtype mice, wt; genetically obese mice, ob/ob fasted mice).

FIG. 7C shows the expression of VDM2 in mice under a high fat (palmitat) diet compared to mice fed a normal diet.

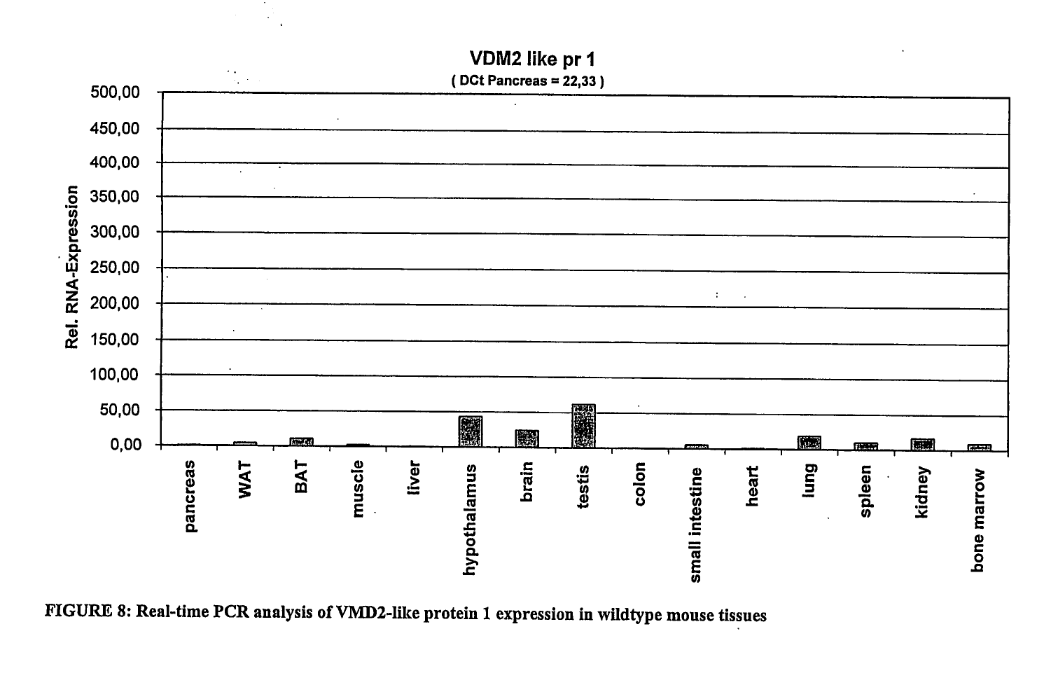

FIG. 8 shows the expression profiling of mouse vitelliform macular dystrophy 2-like protein 1 mRNA.

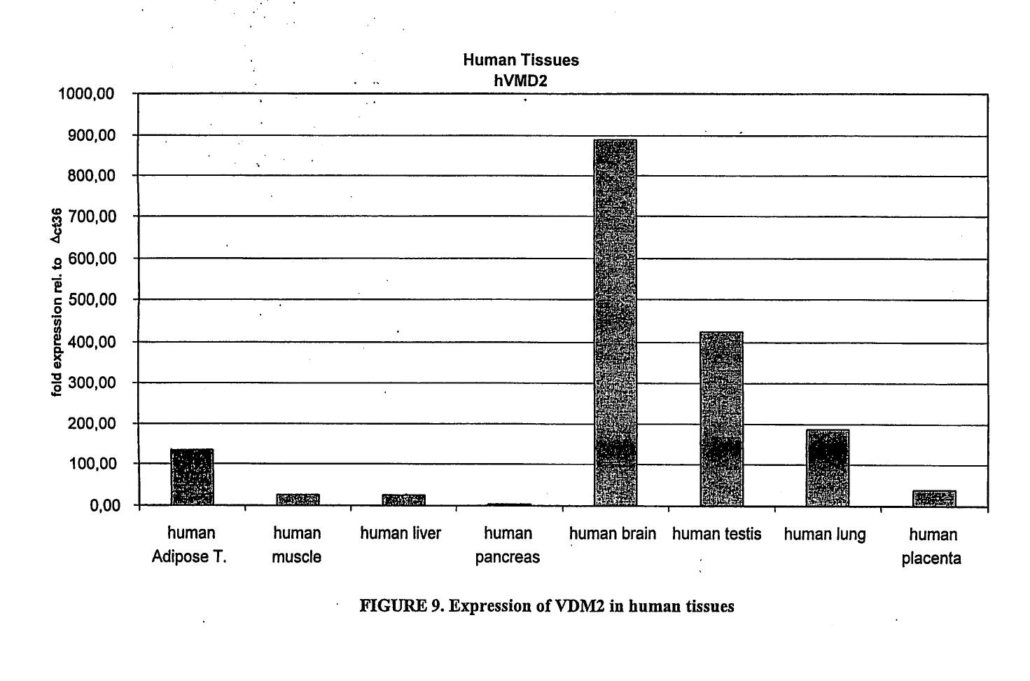

FIG. 9 shows the expression of VMD2 mRNA in different human tissues.

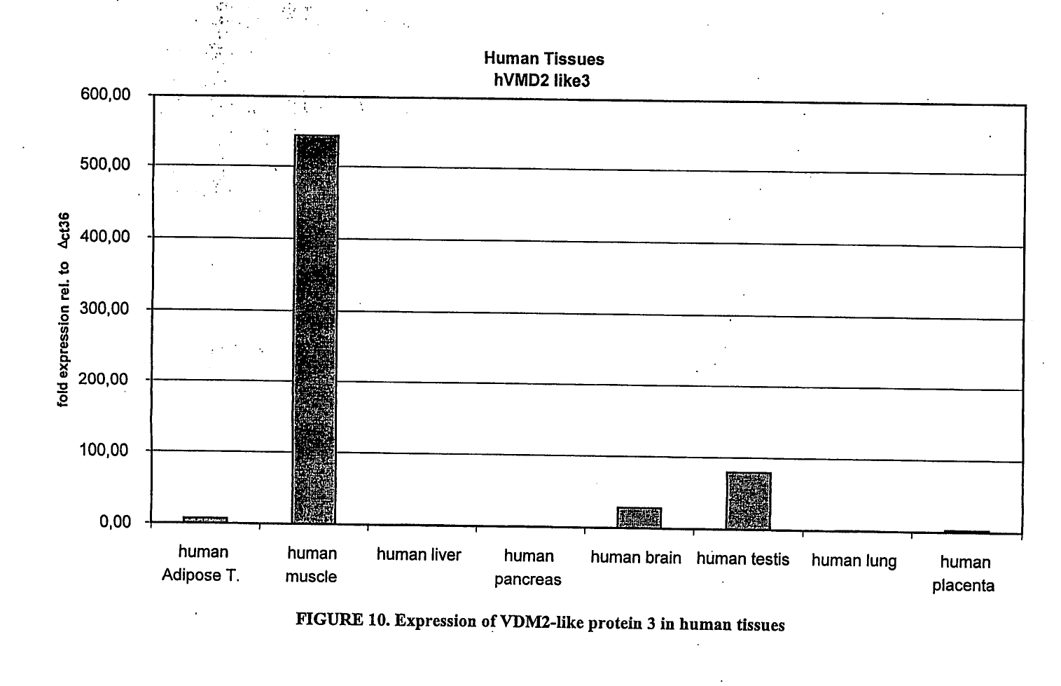

FIG. 10 shows the expression of VMD2-like protein 3 mRNA in different human tissues.

### THE EXAMPLES ILLUSTRATE THE INVENTION

### Example 1

### Measurement of Triglyceride Content of Mutated Flies

The average increase of triglyceride content of homozygous HD-EP(3)32517 and HD-EP(3)36237 flies was investigated in comparison to control flies (FIG. 1). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol. The assay was repeated several times.

The average triglyceride level of EP collection is shown as 100% in FIG. 1. HD-EP(3)32517 homozygous flies show constantly a higher triglyceride content than the controls (260%). In addition HD-EP(3)36237 homozygous flies also show constantly a higher triglyceride content than the controls (160%). Therefore, the loss of gene activity in the locus 85F13-85F14 (estimated), where the EP-vector of HD-EP(3)32517 and HD-EP(3)36237 flies is homozygous viably integrated, is responsible for changes in the metabolism of the energy storage triglycerides, therefore representing in both cases an obese fly model.

### Example 2

### Identification of the Bestrophin Gene

In FIG. 2, genomic DNA sequence is represented by the assembly as a dotted black line (from position 5985465 to 5997965 on chromosome 3R) that includes the integration sites of HD-EP(3)32517 and HD-EP(3)36237. Transcribed DNA sequences (ESTs) and predicted exons are shown as bars in the lower two lines. Predicted exons of gene CG6264 encoding Bestrophin (Best1; GadFly release 3, Bestrophin 1) are shown as dark grey bars and introns as light grey bars. Bestrophin encodes for a gene that is predicted by GadFly sequence analysis programs as CG6264′ Genbank Accession Number NP_{—}652603.1). Using plasmid rescue method genomic DNA sequences that are directly localised 5′ or 3′ of the HD-EP(3)32517 and HD-EP(3)36237 integration site were isolated. Public sequence databases were screened ‘thereby ’ identifying the integration site of HD-EP(3)32517 and HD-EP(3)36237 causing an increase of triglyceride content. HD-EP(3)32517 is integrated in the 5′ exon of the gene Best1 (CG6264) and HD-EP(3)36237 is integrated into the enhancer/promoter region 5′ of Best1 (CG6264). Therefore, expression of the gene Best1 (CG6264) could be effected by homozygous viable integration of HD-EP(3)32517 and HD-EP(3)36237 leading to increase of the energy storage triglycerides.

### Example 3

### Identification of Human Bestrophin Homologues

Bestrophin homologous proteins and nucleic acid molecules coding therefore are obtainable from insect or vertebrate species, e.g. mammals or birds. Particularly preferred are human Bestrophin homologous nucleic acids and polypeptides encoded thereby, particularly encoding (i) a human Bestrophin protein on chromosome 11 (or also refered to as human vitelliform macular dystrophy, VMD2; Genbank Accession No. NP_{—}004174; Swiss Prot. Accession Number 076090; formerly Genbank Accession No. XM_{—}043405, see FIGS. 4A and 4B; SEQ ID NO. 1/2), or (ii) a Bestrophin homologous protein on human chromosome 12 (genomic sequence Genbank Accession No. AC016153, see FIGS. 4E and 4F; SEQ ID NO. 5/6); identical to human vitelliform macular dystrophy 2-like protein 3; Genbank Accession Number AF440758_{—}1, (iii) a Bestrophin homologous protein on human chromosome 1 (genomic sequence Genbank Accession No. AL592166, see FIGS. 4G and 4H; SEQ ID NO. 7/8) identical to encoding a human vitelliform macular dystrophy 2-like protein 2; Genbank Accession Number AF440757_{—}1) or (iv) encoding a Bestrophin homologous protein on human chromosome 19 (assembled from cDNA Genbank Accession No. NM_{—}017682 and genomic sequence AC018761, see FIGS. 4C and 4D or 4I and 4J; SEQ ID NO. 3/4; similar to human vitelliform macular dystrophy 2-like protein 1 (Genbank Accession Number AF440756_{—}1) which has an additional 43 amino acids at the amino terminus). An alignment of Bestrophin from different species has been done by the ClustaW program and is illustrated in FIG. 3 and FIG. 5.

### Example 4

### Expression of the Polypeptides in Mammalian Tissues

For analyzing the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 percent humidity and a light/dark cycle of preferrably 14/10 hours. The mice were fed a standard chow (for example, from ssniff Spezialitäten GmbH, order number ssniff M-Z V1126-000). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). At various time points of the differentiation procedure, beginning with day 0 (day of confluence) and day 2 (hormone addition, for example, dexamethason and 3-isobutyl-1-methylxanthin), up to 10 days of differentiation, suitable aliquots of cells were taken every two days.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH− Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis was performed preferrably using the following primer/probe pairs:

For the amplification of mouse vitelliform macular dystrophy protein (VMD; mouse EnsEMBL Genscan predicted pep tide 19.9000001-10000000.20.329852.331536):

Mouse VMD protein forward primer (SEQ ID NO: 9):
5′-AAG GCC TAT CTT GGA GG TCG A-3′;
mouse VMD protein reverse primer (SEQ ID NO: 10):
5′-GTA CAC ACC TCA TTC ATC AGG CTC-3′;
Taqman probe (SEQ ID NO: 11):
(5/6-FAM) TCC GGG ACA CCG TCC TGC TCC (5/6-TAMRA)

For the amplification of mouse vitelliform macular dystrophy 2-like protein 1 (VMD2L1; GenBank Accession Number NP_{—}663363 or ENSEMBL GenBank Accession Number ENSMUSP00000005276):

Mouse VMD2L1 forward primer (SEQ ID NO: 12):
5′-GCG CTA CGC AGG GCT CT-3′;
mouse VMD2L1 reverse primer (SEQ ID NO: 13):
5′-CCG TTT GAA GAC TGC TGT GC-3′;
Taqman probe (SEQ ID NO: 14):
(5/6-FAM) CGC GGT GCT GAT CCT TCG TTC TG (5/6-
TAMRA)

For the amplification of vitelliform macular dystrophy 2-like protein 3 (VMD2L3; ENSEMBL Accession Number ENSMUSP00000020378):

Mouse VMD2L3 forward primer (SEQ ID NO: 15):
5′-AGG TGG AGC CTG CCA GAG T-3′;
mouse VMD2L3 reverse primer (SEQ ID NO: 16):
5′-AGG ATC TGG ACC TAA GTT TCC C-3′;
Taqman probe (SEQ ID NO: 17):
(5/6-FAM) TCT GGA GTC CAG GCA CAC CTC GC (5/6-
TAMRA).

As shown in FIG. 6A, real time PCR (Taqman) analysis of the expression of the VMD2 like protein 3 mRNA in mammalian (mouse) tissues revealed that VMD2 like protein 3 mRNA is expressed more ubiquitously in different mammalian tissues with clear expression in white adipose tissue (WAT) and brown adipose tissue (BAT). In addition, there are higher levels of expression in muscle, testis, heart, lung, kidney, hypothalamus, and brain tissues. Thus, our analysis shows clearly that the expression of VMD2 like protein 3 mRNA is under metabolic control. In genetically obese (ob/ob) mice (mice without leptin expression), the expression level of VMD2 like protein 3 mRNA is prominently down-regulated (85%) in WAT compared to wildtype levels (FIG. 6B). In addition, the down-regulation in the VMD2 like protein 3 mRNA expression in the WAT of ob/ob mice is also seen in the genetically obese mouse model db/db where the leptin-receptor is missing (see FIG. 6C). Expression of VMD2 like protein 3 mRNA is strongly induced (24 fold upregulation) in the liver of fasted mice (FIG. 6B). In high fat (palmitat) diet mice, the level of VMD2 like protein 3 mRNA is significantly (86%) reduced in WAT compared to mice fed a standard diet (FIG. 6D). During the differentiation of mammalian fibroblast cells (for example, 3T3-L1) from pre-adipocytes to mature adipocytes, the expression of VDM2 like protein 3 is significantly (81%) downregulated (FIG. 6E). This could indicate that VDM2-like protein 3 is an inhibitor of adipocyte lipid accumulation.

As shown in FIG. 7A, real time PCR (Taqman) analysis of the expression of the VMD2 protein mRNA in mammalian (mouse) tissues revealed that VMD2 mRNA is expressed ubiquitously in different mammalian tissues, with clear expression in adipose tissues (WAT and BAT) (FIG. 7A). Our results clearly show that the expression of VDM2 mRNA is under metabolic control. In genetically obese (ob/ob) mice, expression of VDM2 mRNA is strongly induced in WAT (FIG. 7B). In mice under a high fat diet, a strong upregulation of VDM2 mRNA can be observed in muscle (11 fold) and in BAT, see FIG. 7C.

As shown in FIG. 8, real time PCR (Taqman) analysis of the expression of the VMD2 like protein 1 protein mRNA in mammalian (mouse) tissues revealed that VMD2 like protein 1 mRNA is expressed in different mammalian tissues, showing highest level of expression in colon and higher levels in hypothalamus, brain and testis.

No results are shown for VDM2 like protein 2.

Although all proteins are expressed in the hypothalamus and to a lesser degree in other brain areas they show a clear tissue specific expression suggesting distinct roles in the different metabolic requirements of various tissues.

### Example 5

### Expression of the Polypeptides in Mammalian (Human) Tissues

For analyzing the expression of the polypeptides disclosed in this invention in mammalian tissues, human RNAs isolated from different tissues were obtained from Invitrogen Corp., Karlsruhe, Germany: (i) total RNA from human adult skeletal muscle (Invitrogen Corp. Order Number 735030); (ii) total RNA from human adult lung (Invitrogen Corp. Order Number 735020); (iii) total RNA from human adult liver (Invitrogen Corp. Order Number 735018); (iv) total RNA from human adult placenta (Invitrogen Corp. Order Number 735026); (v) total RNA from human adult testis (Invitrogen Corp. Order Number 64101-1); (vi) total RNA from human normal adipose tissue (Invitrogen Corp. Order Number D6005-01); (vii) total RNA from human normal pancreas (Invitrogen Corp. Order Number DG6101); (viii) total RNA from human normal brain (Invitrogen. Corp. Order Number D6030-01).

The RNA was treated with DNase according to the instructions of the manufacturers (for example, from Qiagen, Germany) and as known to those skilled in the art. Total RNA was reverse transcribed (preferrably using Superscript II RNaseH− Reverse Transcriptase, from Invitrogen, Karlsruhe, Germany) and subjected to Taqman analysis preferrably using the Taqman 2×PCR Master Mix′ (from Applied Biosystems, Weiterstadt, Germany). The Taqman 2×PCR Master Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (all obtained from Applied Biosystems, Weiterstadt, Germany).

Taqman analysis was performed preferrably using the following primer/probe pairs:

For the amplification of VMD2:

human VMD2 forward primer (SEQ ID NO: 18):
5′-TCA CGC TGG CAT CAT TGG A-3′;
human VMD2 reverse primer (SEQ ID NO: 19):
5′-CCC TGG GAG GAT GG TGA TC-3′;
Taqman probe (SEQ ID NO: 20):
(5/6-FAM) CGC TTC CTA GGC CTG CAG TCC CA (5/6-
TAMRA)

For the amplification of VMD2 like protein 3:

human VMD2 like3 forward primer (SEQ ID NO: 21):
5′-CCC ACC ATA CAC ATT GGC AG-3′;
human VMD2 like3 reverse primer (SEQ ID NO: 22):
5′-TTT CCC CAT CTG GAC TGT TGA-3′;
Taqman probe (SEQ ID NO: 23):
(5/6-FAM) TGC TGA CTA CTG CAT ACC CTC ATT TCT GGG
T (5/6-TAMRA)

For the amplification of VMD2 like protein 1:

human VMD2 like1 forward primer (SEQ ID NO: 24):
5′-AGG TCC CTG CAC GGC A-3′;
human VMD2 like1 reverse primer (SEQ ID NO: 25):
5′-TTT ACA AAG GCA CAC GAG GCT-3′;
Taqman probe (SEQ ID NO: 26):
(5/6-FAM) CCA CGC AGG TGT CCC GGT CTG (5/6-TAMRA)

For the amplification of VMD2 like protein 2:

human VMD2 like2 forward primer (SEQ ID NO: 27):
5′-CAT CAC GGA AGG TCT TGT CAA A-3′;
human VMD2 like2 reverse primer (SEQ ID NO: 28):
5′-TCC CAA ATC TAA CGT GCC AGA-3′;
Taqman probe (SEQ ID NO: 29):
(5/6-FAM) TGC TGG GCA CCA CTC CCA GCA T (5/6-
TAMRA)

As shown in FIG. 9, real time PCR (Taqman) analysis of the expression of VDM 2 protein in human tissues revealed that VDM2 is expressed in different mammalian tissues, showing higher levels of expression in brain, testis, lung and adipose tissue, suggesting a role in the regulation of energy homeostasis. The data obtained with human tissue are in good agreement with the data obtained with mouse tissues (see FIG. 7A). Particularly, an upregulation of VMD2 in human preadipocytes was found.

As shown in FIG. 10, real time PCR (Taqman) analysis of the expression of VDM2 like protein 3 in human tissues revealed that VDM2 like protein 3 is expressed in different human tissues, showing highest level of expression in muscle and to a lesser extend in brain and testis. Similar results were obtained with mouse tissues (see FIG. 6A).

Not shown are the results for VDM2 like protein 1 and VDM2 like protein 2 as these genes showed no detectable expression in the tissues analyzed so far.

1. A pharmaceutical composition comprising a nucleic acid molecule of the Bestrophin gene family or a polypeptide encoded thereby or a fragment or a variant of said nucleic acid molecule or said polypeptide or an antibody, an aptamer or another receptor recognizing a nucleic acid molecule of the Bestrophin gene family or a polypeptide encoded thereby together with pharmaceutically acceptable carriers, diluents and/or adjuvant.

2. The composition of claim 1, wherein the nucleic acid molecule is a vertebrate or insect Bestrophin nucleic acid, particularly a human Bestrophin nucleic acid (i) encoding a Bestrophin homologous protein 15 on human chromosome 12 (SEQ NO. 5/6); referred to as human vitelliform macular dystrophy 2-like protein 3, (ii) encoding a human Bestrophin protein on human chromosome 11 (SEQ 10 NO.1/2); referred to as human vitelliform macular dystrophy, VMD2, (iii) encoding a Bestrophin homologous protein on human chromosome 1 (SEQ 10 NO. 7/8); referred to as human vitelliform macular dystrophy 2-like protein 2 or (iv) encoding a Bestrophin homologous protein on human chromosome 19 (SEQ 10 NO. 3/4); referred to a (human vitelliform macular dystrophy 2-like protein 1 or a fragment thereof or a variant thereof.

3. The composition of claim 1 or 2, wherein said nucleic acid molecule comprises
(a) a Bestrophin nucleotide sequence as shown in FIG. 4 or the complementary strand thereof,
(b) a nucleotide sequence which hybridizes under stringent conditions to a nucleic acid molecule encoding a Bestrophin amino acid sequence as shown in FIG. 4 or the complementary strand thereof,
(c) a nucleotide sequence corresponding to the sequences of (a) or (b) within the degeneration of the genetic code,
(d) a nucleotide sequence which encodes a polypeptide which is at least 85%, preferably at least 90%, more preferably at least 95%, more preferably at least 98% and up to 99.6% identical to the amino acid sequences shown in FIG. 4,
(e) a nucleotide sequence which differs from the nucleic, acid molecule of (a) to (d) by mutation and wherein said mutation causes an alteration, deletion, duplication or premature stop in the encoded polypeptide or
(f) a partial nucleotide sequence of any of the sequences of (a) to (e) having a length of at least 15 bases, preferably at least 20 bases, more preferably at least 25 bases and most preferably at least 50 bases.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting and/or verifying, for the treatment, alleviation and/or prevention of an disorders, including metabolic diseases such as obesity and other body-weight regulation disorders as well as related disorders such as eating disorder, cachexia, diabetes mellitus, hypertension, coronary heart disease, hypercholesterolemia, osteoarthritis, gallstones, cancers of the reproductive organs, and sleep apnea and others, in cells, cell masses, organs and/or subjects.

14. Use of a nucleic acid molecule of the Bestrophin gene family or a polynucleotide encoded thereby or a fragment or a variant of said nucleic acid molecule or said polypeptide or an antibody, an aptamer or another receptor recognizing a nucleic acid molecule of the Bestrophin gene family or a polypeptide encoded thereby for controlling the function of a gene and/or a gene product which is influenced and/or modified by a Bestrophin homologous polypeptide.

15. Use of the nucleic acid molecule of the Bestrophin gene family or a polynucleotide encoded thereby or a fragment or a variant of said nucleic acid molecule or said polypeptide or an antibody, an aptamer or another receptor recognizing a nucleic acid molecule of the Bestrophin gene family or a polypeptide encoded thereby for identifying substances capable of interacting with a Bestrophin homologous polypeptide.

16. A non-human transgenic animal exhibiting a modified expression of a Bestrophin homologous polypeptide.

17. The animal of claim 16, wherein the expression of the Bestrophin homologous polypeptide is increased and/or reduced.

18. A recombinant host cell exhibiting a modified expression of an Bestrophin homologous polypeptide.

19. The cell of claim 18 which is a human cell.

20. A method of identifying a (poly)peptide involved in the regulation of energy homeostasis and/or metabolism of triglycerides in a mammal comprising the steps of
(a) contacting .a collection of (poly)peptides with a Bestrophin homologous polypeptide or a fragment thereof under conditions that allow binding of said (poly) peptides;
(b) removing (poly)peptides which do not bind and
(c) identifying (poly)peptides that bind to said Bestrophin homologous polypeptide.

21. A method of screening for an agent which modulates the interaction of a Bestrophin homologous polypeptide with a binding target/agent, comprising the steps of
(a) incubating a mixture comprising
(aa) a Bestrophin homologous polypeptide, or a fragment thereof;
(ab) a binding target/agent of said Bestrophin homologous polypeptide or fragment thereof; and
(ac) a candidate agent.
under conditions whereby said Bestrophin polypeptide or fragment thereof specifically binds to said binding target/agent at a reference affinity;
(b) detecting the binding affinity of said Bestrophin polypeptide or fragment thereof to said binding target to determine an (candidate) agent-biased affinity; and
(c) determining a difference between (candidate) agent-biased affinity and the reference affinity.

22. A method of screening for an agent which modulates the activity of a Bestrophin homologous polypeptide, comprising the steps:
(a) incubating a mixture comprising
(aa) a Bestrophin homologous polypeptide, or a fragment thereof;
(ab) a candidate agent;
under conditions whereby an activity of said Bestrophin polypeptide or fragment thereof may be determined;
(b) determining the activity of said Bestrophin polypeptide or fragment thereof in the presence of said candidate agent; and
(c) determining a difference between the activity in presence of said candidate agent and a reference activity.

23. The method of claim 22 wherein the activity is selected from a chloride channel or another conductance activity.

24. The method of claim 22 wherein the activity is selected from the degree of phosphorylation or other posttranslational modifications.

25. A pharmaceutical composition comprising the (poly)peptide identified by the method of claim 20 with a pharmaceutically acceptable carrier, diluent and/or adjuvant.

26. The composition of claim 25 wherein said composition is a pharmaceutical composition for preventing, alleviating or treating of diseases and disorders, including metabolic diseases such as obesity and other body-weight regulation disorders as well as related disorders such as eating disorder, cachexia, diabetes mellitus, hypertension, coronary heart disease, hypercholesterolemia, osteoarthritis, gallstones, cancers of the reproductive organs, and sleep apnea and other diseases and disorders.

27. Use of a (poly)peptide as identified by the method of claim 20 for the preparation of a pharmaceutical composition for the treatment, alleviation and/or prevention of diseases and disorders, including metabolic diseases such as obesity and other body-weight regulation disorders as well as related disorders such as eating disorder, cachexia, diabetes mellitus, hypertension, coronary heart disease, hypercholesterolemia, osteoarthritis, gallstones, cancers of the reproductive organs, and sleep apnea and other diseases and disorders.

28. Use of a nucleic acid molecule of the Bestrophin family or of a fragment thereof for the preparation of a non-human animal which over- or underexpresses the Bestrophin gene product.

29. Kit comprising at least one of
(a) an Bestrophin nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

30. A pharmaceutical composition comprising the agent identified by the method of claim 21 with a pharmaceutically acceptable carrier, diluent and/or adjuvant.

31. Use of an agent as identified by the method of claim 21 for the preparation of a pharmaceutical composition for the treatment, alleviation and/or prevention of diseases and disorders, including metabolic diseases such as obesity and other body-weight regulation disorders as well as related disorders such as eating disorder, cachexia, diabetes mellitus, hypertension, coronary heart disease, hypercholesterolemia, osteoarthritis, gallstones, cancers of the reproductive organs, and sleep apnea and other diseases and disorders.


SEQUENCE LISTING (37 sequences)

SEQ ID NO: 1 - DNA, 1758 bp, Homo sapiens
atgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacacccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaa

SEQ ID NO: 2 - protein, 585 aa, Homo sapiens
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 3 - DNA, 2137 bp, Homo sapiens
atgaaggtcagggaggggcactgggggctggactagactgagtaaagctgggaggagaaggcaactcaggcaagggtcaggactgaaggggtggcgggccggaacatgggtggaatccctgtgctcagttatccccgcacctctccacccacacccgcagcccccacccgggccacccactctcccttggccacacctgccgggtgccacgatgaccgtcacctacacagcccgagtggcgaacgcccgcttcggtggcttctcccagctgctgctactgtggcgtgggagcatctacaaactcctgtggcgagagctgctctgcttccttgggttctacatggcgctgagtgctgcctaccgctttgtgctgaccgaagggcagaagcgctacttcgagaagcttgtgatttattgtgaccagtatgccagcctcatccctgtctccttcgtgcttggcttttatgtgacgctggtggtgaaccgctggtggagccagtacctatgcatgccgctgcccgacgcgctcatgtgcgtggtggcgggcaccgtgcacggacgcgacgaccgcggccgcctctaccggcgcacactcatgcgctacgcagggctctcggccgtgctcatcctgcgctccgtcagcaccgcggtgttcaagcgcttccccaccatagaccacgtggtggaggctgggtttatgacccgcgaggagcgcaagaagtttgaaaacctgaactcatcctacaacaagtactgggtgccctgcgtctggttctccaacctggcggcacaggcccgacgcgagggccgcatccgcgacaacagcgcccttaagctgctgctcgaggagctgaatgtttttcggggcaaatgtggaatgctctttcactatgactggattagcgtacccctcgtgtacacgcaggtggtgaccatcgcactgtacagctacttcctggcttgcctcattggtcgccagttcctggacccggctcagggttacaaagaccacgacctagacctgtgtgtgcccatcttcaccctcttgcagttcttcttctacgccggctggctcaaggtagctgagcagctcatcaaccccttcggagaggacgatgatgactttgagaccaactttctgatcgatagaaacttccaggtgtccatgctggcagtggacgagatgtatgatgacctggctgtgctggagaaggacttgtactgggatgcagccgaggctcgcgccccatacacagcggctactgtcttccagctgcggcagccttccttccagggctccacctttgacatcacgctggccaaagaagacatgcagttccagcggctggacggcttggatggaccgatgggaggggcgcccggcgacttcctgcagcgcctcctgccggcgggcgcgggcatggtcgcgggaggcccgctggggcggcgcctgtcctttctactccgcaagaacagctgcgtgtcggaggcgtctactggggccagctgctcatgcgcggttgtccccgaaggcgcggccccggagtgcagctgcggggacccgctgctcgaccccggcctgccggagcccgaggccccgccccctgcgggtcccgaaccgcttaccctcatccctgggcctgtcgagcccttcagcatcgtgaccatgcccgggccccggggtccggcgccaccctggctgcccagccctattggcgaggaggaggagaatctggcctgagatcttagagcccagccccctaaggacagggaaccaggtccctgcacggcacccacgcaggtgtcccggtctgcataagcctcgtatgcctttgtaaagtccacctacacttttgaccagctctcgctgcccgcatgtgtttggcgctgtgctaggggcgggagttcttccagactcttggaccagcccgccctgaccaccagctctacttcccaacccccactgcctgagaggtctctatcagtgtcctgcctgaattctttccttcaagtgaagatgtgactgactacctcctcgagttgtcatgaggatgaaagaatggcattaaagcatttggtatacagtaggtgctcaataaattgctagtgatttttactcaaaaaaaaaaaaaaaaaa

SEQ ID NO: 4 - protein, 509 aa, Homo sapiens [sequence omitted]

SEQ ID NO: 5 - DNA, 2028 bp, Homo sapiens
atgactgtcacttactccagtaaagtagcaaatgcaactttttttggatttcataggttactcctcaagtggagaggcagcatctacaaactactgtacagggaatttattgtttttgctgttctttatacagcaataagtttggtatacagattgttacttacaggagtccaaaaacgttactttgaaaaattatcaatttactgtgacagatatgctgaacaaattccagtaacctttgtgcttgggttttatgttactctggtagtgaaccgatggtggaaccagtttgtgaatttgccctggccagacaggctaatgttcctcatctctagcagtgttcacggaagcgacgagcacgggcgcctgcttagaaggacgctgatgcgctacgtcaatctcacctccctgctcatctttcgctcggtgagcactgctgtgtacaaaagatttcccacaatggaccacgtggttgaagcaggttttatgacaacagatgaaaggaaattattcaaccacctcaagtctcctcatctgaaatattgggttccattcatctggtttggaaatcttgcaactaaagcccggaatgaaggtagaatcagagacagtgttgatctgcaatcattgatgactgaaatgaatcgataccgctcttggtgcagcctcttattcggttatgactgggttgggattccgctggtttacacccaggttgtcactcttgctgtctataccttcttctttgcgtgcctgattggacgccagtttttggatcccaccaaaggctacgcagggcatgacttggatctttacattcccatcttcaccctcctacaattcttcttctatgcaggatggcttaaggattataataatgtaactaaagtagtagcagagcagcttatcaacccttttggagaagatgatgatgattttgaaactaactggtgcattgacagaaatttgcaggtctctcttttagctgtggacgaaatgcacatgagcttacccaagatgaagaaggacatttactgggacgattctgctgctcgcccaccatacacattggcagctgctgactactgcataccctcatttctggggtcaacagtccagatgctgtctgggtccgactttcctgacgaggagtggctgtgggattatgagaagcatggccatcggcattccatgataagaagagtcaagcggttcctgagtgcccacgaacacccctccagccccagaagaagaagctacaggaggcagacaagtgacagctccatgttcttaccccgagatgacctcagcccagccagggacctactggatgtgccctcaagaaacccccccagggcctcacccacctggaagaaatcctgcttcccagaaggaagccccacgctgcacttcagcatgggagagctgtccaccatcagggagaccagccagacaagcactttacagagcctgaccccacagtccagtgtgagaacttcccccatcaaaatgccactggtacctgaggtattgatcacagcagccgaagcaccagtgcccacatcagggggctaccaccatgattccgctacctccatcttgagctctgagtttacaggggttcagccaagcaagactgagcagcagcagggccccatgggatccatcctgtctccctcagagaaggagacacctcctggaggccccagtccccagacagtttcagccagcgctgaggaaaatatattcaactgtgaagaagaccctggtgatacctttctaaaaaggtggagtcttccgggattcctggggtccagccacacttccctgggaaacctaagtccagaccccatgagctctcagccagctcttttaattgacacagaaacatcctcagagatcagtgggatcaacattgtggctggctctcgagtctcttctgatatgctgtatttaatggaaaacctggacaccaaggaaacagatatcatagagctgaacaaggaaactgaggaatcacccaaatga

SEQ ID NO: 6 - protein, 675 aa, Homo sapiens
MTVTYSSKVANATFFGFHRLLLKWRGSIYKLLYREFIVFAVLYTAISLVYRLLLTGVQKRYFEKLSIYCDRYAEQIPVTFVLGFYVTLVVNRWWNQFVNLPWPDRLMFLISSSVHGSDEHGRLLRRTLMRYVNLTSLLIFRSVSTAVYKRFPTMDHVVEAGFMTTDERKLFNHLKSPHLKYWVPFIWFGNLATKARNEGRIRDSVDLQSLMTEMNRYRSWCSLLFGYDWVGIPLVYTQVVTLAVYTFFFACLIGRQFLDPTKGYAGHDLDLYIPIFTLLQFFFYAGWLKDYNNVTKVVAEQLINPFGEDDDDFETNWCIDRNLQVSLLAVDEMHMSLPKMKKDIYWDDSAARPPYTLAAADYCIPSFLGSTVQMLSGSDFPDEEWLWDYEKHGHRHSMIRRVKRFLSAHEHPSSPRRRSYRRQTSDSSMFLPRDDLSPARDLLDVPSRNPPRASPTWKKSCFPEGSPTLHFSMGELSTIRETSQTSTLQSLTPQSSVRTSPIKMPLVPEVLITAAEAPVPTSGGYHHDSATSILSSEFTGVQPSKTEQQQGPMGSILSPSEKETPPGGPSPQTVSASAEENIFNCEEDPGDTFLKRWSLPGFLGSSHTSLGNLSPDPMSSQPALLIDTETSSEISGINIVAGSRVSSDMLYLMENLDTKETDIIELNKETEESPK

SEQ ID NO: 7 - DNA, 1422 bp, Homo sapiens
atgacggtttcatacactctcaaagtggcggaggcccgcttcggaggtttctctggcctgcttctccgctggaggggaagcatctacaagctcctctacaaggaattcctcctctttggggccttgtacgctgtgcttagcatcacctaccggctgctgctgacccaggagcagaggtacgtgtatgctcaggtggcccggtactgcaaccgctcagcagacctcattcccttgtcctttgtattgggtttctatgtgactctcgtggtgaaccgctggtggtcccagtacacaagcatcccgctgccagaccagctgatgtgcgtcatctcggctagcgtgcacggcgtggaccagcggggccgcctgctgcgccgcaccctcatccgctacgcgaacctggcgtccgtgctggtgctgcgctcggtcagcacccgcgtgcttaagcgcttccccaccatggagcacgtggtggacgcaggtttcatgtcccaggaagagaggaaaaagtttgagagcctgaaatccgacttcaacaagtactgggtcccctgcgtctggttcaccaacctggcggcccaggcccggagggacgggcgaatacgtgacgatatcgctctctgtctacttttggaagagctgaacaagtaccgagccaagtgcagcatgctattccactatgactggatcagcatccccctcgtctacacccaagtggtgaccatagccgtctactctttctttgccctctccctggttggccgccagtttgtggagccagaggcaggggctgccaaacctcagaagcttctgaagccaggccaggagccagccccagccctgggagacccggacatgtacgtgcctctcaccactctgctgcagttcttcttctatgctggctggctcaaggtggctgaacagatcatcaacccatttggtgaggatgatgacgactttgagacaaatcagctcatagaccgcaacttgcaggtgtccctgctatccgtggacgaaatgtaccagaaccttccccccgctgagaaggaccagtactgggatgaggaccagccgcagccaccctacactgtggccacggcggccgagtctctgcggccctcattcctgggctccaccttcaacctgcgcatgagcgacgaccctgagcagagcctgcaggtggaggcgtcccccggatctggtcggcccgcgcccgccgcgcagaccccgttgctcggccgcttcctgggcgtaggggcgccctccccggccatcagcctccggaacttcggccgcgtgcgaggcaccccccgccccccgcatctgctgcgcttccgggcggaggagggcggcgaccccgaggccgcagcccgcatcgaggaggaatcggcggagtccggggacgaggccctggagccctga

SEQ ID NO: 8 - protein, 473 aa, Homo sapiens
MTVSYTLKVAEARFGGFSGLLLRWRGSIYKLLYKEFLLFGALYAVLSITYRLLLTQEQRYVYAQVARYCNRSADLIPLSFVLGFYVTLVVNRWWSQYTSIPLPDQLMCVISASVHGVDQRGRLLRRTLIRYANLASVLVLRSVSTRVLKRFPTMEHVVDAGFMSQEERKKFESLKSDFNKYWVPCVWFTNLAAQARRDGRIRDDIALCLLLEELNKYRAKCSMLFHYDWISIPLVYTQVVTIAVYSFFALSLVGRQFVEPEAGAAKPQKLLKPGQEPAPALGDPDMYVPLTTLLQFFFYAGWLKVAEQIINPFGEDDDDFETNQLIDRNLQVSLLSVDEMYQNLPPAEKDQYWDEDQPQPPYTVATAAESLRPSFLGSTFNLRMSDDPEQSLQVEASPGSGRPAPAAQTPLLGRFLGVGAPSPAISLRNFGRVRGTPRPPHLLRFRAEEGGDPEAAARIEEESAESGDEALEP

SEQ ID NO: 9 - DNA, 21 bp, Artificial
  primer
aaggcctatcttggaggtcga

SEQ ID NO: 10 - DNA, 24 bp, Artificial
  primer
gtacacacctcattcatcaggctc

SEQ ID NO: 11 - DNA, 21 bp, Artificial
  Taqman probe
tccgggacaccgtcctgctcc

SEQ ID NO: 12 - DNA, 17 bp, Artificial
  primer
gcgctacgcagggctct

SEQ ID NO: 13 - DNA, 20 bp, Artificial
  primer
ccgtttgaagactgctgtgc

SEQ ID NO: 14 - DNA, 23 bp, Artificial
  Taqman probe
cgcggtgctgatccttcgttctg

SEQ ID NO: 15 - DNA, 19 bp, Artificial
  primer
aggtggagcctgccagagt

SEQ ID NO: 16 - DNA, 22 bp, Artificial
  primer
aggatctggacctaagtttccc

SEQ ID NO: 17 - DNA, 23 bp, Artificial
  Taqman probe
tctggagtccaggcacacctcgc

SEQ ID NO: 18 - DNA, 19 bp, Artificial
  primer
tcacgctggcatcattgga

SEQ ID NO: 19 - DNA, 19 bp, Artificial
  primer
ccctgggaggatggtgatc

SEQ ID NO: 20 - DNA, 23 bp, Artificial
  Taqman probe
cgcttcctaggcctgcagtccca

SEQ ID NO: 21 - DNA, 20 bp, Artificial
  primer
cccaccatacacattggcag

SEQ ID NO: 22 - DNA, 21 bp, Artificial
  primer
tttccccatctggactgttga

SEQ ID NO: 23 - DNA, 31 bp, Artificial
  Taqman probe
tgctgactactgcataccctcatttctgggt

SEQ ID NO: 24 - DNA, 16 bp, Artificial
  primer
aggtccctgcacggca

SEQ ID NO: 25 - DNA, 21 bp, Artificial
  primer
tttacaaaggcacacgaggct

SEQ ID NO: 26 - DNA, 21 bp, Artificial
  Taqman probe
ccacgcaggtgtcccggtctg

SEQ ID NO: 27 - DNA, 22 bp, Artificial
  primer
catcacggaaggtcttgtcaaa

SEQ ID NO: 28 - DNA, 21 bp, Artificial
  primer
tcccaaatctaacgtgccaga

SEQ ID NO: 29 - DNA, 22 bp, Artificial
  Taqman probe
tgctgggcaccactcccagcat

SEQ ID NO: 30 - DNA, 1530 bp, Homo sapiens
atgaccgtcacctacacagcccgagtggcgaacgcccgcttcggtggcttctcccagctgctgctactgtggcgtgggagcatctacaaactcctgtggcgagagctgctctgcttccttgggttctacatggcgctgagtgctgcctaccgctttgtgctgaccgaagggcagaagcgctacttcgagaagcttgtgatttattgtgaccagtatgccagcctcatccctgtctccttcgtgcttggcttttatgtgacgctggtggtgaaccgctggtggagccagtacctatgcatgccgctgcccgacgcgctcatgtgcgtggtggcgggcaccgtgcacggacgcgacgaccgcggccgcctctaccggcgcacactcatgcgctacgcagggctctcggccgtgctcatcctgcgctccgtcagcaccgcggtgttcaagcgcttccccaccatagaccacgtggtggaggctgggtttatgacccgcgaggagcgcaagaagtttgaaaacctgaactcatcctacaacaagtactgggtgccctgcgtctggttctccaacctggcggcacaggcccgacgcgagggccgcatccgcgacaacagcgcccttaagctgctgctcgaggagctgaatgtttttcggggcaaatgtggaatgctctttcactatgactggattagcgtacccctcgtgtacacgcaggtggtgaccatcgcactgtacagctacttcctggcttgcctcattggtcgccagttcctggacccggctcagggttacaaagaccacgacctagacctgtgtgtgcccatcttcaccctcttgcagttcttcttctacgccggctggctcaaggtagctgagcagctcatcaaccccttcggagaggacgatgatgactttgagaccaactttctgatcgatagaaacttccaggtgtccatgctggcagtggacgagatgtatgatgacctggctgtgctggagaaggacttgtactgggatgcagccgaggctcgcgccccatacacagcggctactgtcttccagctgcggcagccttccttccagggctccacctttgacatcacgctggccaaagaagacatgcagttccagcggctggacggcttggatggaccgatgggagaggcgcccggcgacttcctgcagcgcctcctgccggcgggcgcgggcatggtcgcgggaggcccgctgggccggcgcctgtcctttctactccgcaagaacagctgcgtgtcggaggcgtctactggggccagctgctcatgcgcggttgtccccgaaggcgcggccccggagtgcagctgcggggacccgctgctcgaccccggcctgccggagcccgaggccccgccccctgcgggtcccgaaccgcttaccctcatccctgggcctgtcgagcccttcagcatcgtgaccatgcccgggccccggggtccggcgccaccctggctgcccagccctattggcgaggaggaggagaatctggcctga

SEQ ID NO: 31 - protein, 509 aa, Homo sapiens
MTVTYTARVANARFGGFSQLLLLWRGSIYKLLWRELLCFLGFYMALSAAYRFVLTEGQKRYFEKLVIYCDQYASLIPVSFVLGFYVTLVVNRWWSQYLCMPLPDALMCVVAGTVHGRDDRGRLYRRTLMRYAGLSAVLILRSVSTAVFKRFPTIDHVVEAGFMTREERKKFENLNSSYNKYWVPCVWFSNLAAQARREGRIRDNSALKLLLEELNVFRGKCGMLFHYDWISVPLVYTQVVTIALYSYFLACLIGRQFLDPAQGYKDHDLDLCVPIFTLLQFFFYAGWLKVAEQLINPFGEDDDDFETNFLIDRNFQVSMLAVDEMYDDLAVLEKDLYWDAAEARAPYTAATVFQLRQPSFQGSTFDITLAKEDMQFQRLDGLDGPMGEAPGDFLQRLLPAGAGMVAGGPLGRRLSFLLRKNSCVSEASTGASCSCAVVPEGAAPECSCGDPLLDPGLPEPEAPPPAGPEPLTLIPGPVEPFSIVTMPGPRGPAPPWLPSPIGEEEENLA

SEQ ID NO: 32 - protein, 721 aa, Drosophila melanogaster
MTITYTGEVATCRGFGCFLKLLLRWRGSIYKLVWLDLLAFLTIYYAINMVYRFGLNPAQKETFEAIVQYCDSYRELIPLSFVLGFYVSIVMTRWWNQYTSIPWPDPIAVFVSSNVHGQDERGRMMRRTIMRYVCLCLTMVLANVSPRVKKRFPGLNNLVEAGLLNDNEKTIIETMNKAFPRPSKHWLPIVWAASIITRARKEGRIRDDFAVKTIIDELNKFRGQCGLLISYDTISVPLVYTQVVTLAVYSYFLTCCMGQQWTDGKVVGNTTYLNKVDLYFPVFTTLQFFFYMGWLKVAESLINPFGEDDDDFEVNWMVDRNLQVSYLIVDEMHHDHPELLKDQYWDEVFPNELPYTIAAERFRENHPEPSTAKIEVPKNAAMPSTMSSVRIDEMADDASGIHFSAGNGKMRLDSSPSLVSVSGTLSRVNTVASALKRFLSRDDSRPGSATPSQDQPYKFPASASSASLSGAVVGSATSAGKPAGSLRITQQVIEEVDEQATITSMRANDPRPNVMDIFAQTSSGAGTSGPLQPPPAHSEPVDIPSRPPSYNRAQSQYEPNLFPPGGVDALLSTSAPAGGSPLLLSNAATAPSSPVGESSKSLYDPQKGASRETVESMDLRSSTDLLGDAAVQPEDEGDDFDKLKAEREKEKLMRQQKNLARTISTAPGMEATAVPMVPMVPVNVAVQQAQLQPVASSADLLAGGDQFSNSTMKSEDAINGS

SEQ ID NO: 33 - protein, 187 aa, Mus musculus
MVLSSDQQLLFEKLALYCDSYIQLIPISFVLGFYVTLVVSRWWSQYENLPWPDRLMIQVSSFVEGKDEEGRLLRRTLIRYAILGQVLILRSISTSVYKRFPTLHHLVLAGFMTHGEHKQLQKLGLPHNTFWVPWVWFANLSMKAYLGGRIRDTVLLQSLMNEVCTLRTQCGQLYAYDWISIPLVYTQ

SEQ ID NO: 34 - protein, 262 aa, Mus musculus
MTVTYSSKVANATFFGFHRLLLKWRGSIYKLLYREFIVFAVLYTAISLVYRLLLTGAQKRYFEKLSIYCDRYAEQIPVTFVLGFYVTLVVNRWWNQFVNLPWPDRLMLLISSSVHGSDQHGRLLRRTLMRYVNLTSLLIFRSVSTAVYKRFPTMDHVVEAGFMTADERKLFDHLKSPHLKYWVPFIWFGNLATKARNEGRIRDSVDLQSLMTEMNRYRSWCSLLFGYDWVGIPLVYTQVVTLAVYTFFFACLIGRQFLDPTK

SEQ ID NO: 35 - protein, 465 aa, Mus musculus
MALSAAYRFLLAEEQKRYFEKLVIYCDQYASLIPVSFVLGFYVTLVVHRWWNQYLCMPLPDALMCIVAGTVHGRDDRGRLYRRTLMRYAGLSAVLILRSVSTAVFKRFPTIDHVVEAGFMTREERKKFENLNSSYNKYWVPCVWFSSLAAQARREGRIRDNSALKLLLEELNVFRSKCGMLFHYDWISIPLVYTQVVTIAVYSYFLACLIGRQFLDPAQGYKDHTLDLCVPIFTLLQFFFYAGWLKVAEQLINPFGEDDDDFETNFLIDRNFQVSMLAVDEMYDDLAMLEKDLYWDAAEARAPYTAATAFLLQQPSFQGSTFDIALAKEDMQFQRLDGVDGPLGEVHGDFLQRLLPAGAGSVGPLGRRLSLLRRKNSCVSEASTAASCGCAGAADGGGVECGCGDPLLDPSLREPELEPPACPEPPAPIPGPTPEPFTTVSIPGPRAPAPPWLPSPIGEEEESPA

SEQ ID NO: 36 - protein, 142 aa, Mus musculus
WLFATRYANLASVLVLRSVSTRVLKRFPTMEHVVDAGFMSQEERKKFESLKSDFNKYWVPCVWFTNLAAQACRDGRIRDDIALCLILEELNKYRAKCSMLFHYDWISVPLVYTQVTAQLRTSYLLHFQTGDEEAEGRIDEAV

SEQ ID NO: 37 - protein, 398 aa, Homo sapiens
MTVTYSSKVANATFFGFHRLLLKWRGSIYKLLYREFIVFAVLYTAISLVYRLLLTGVQKRYFEKLSIYCDRYAEQIPVTFVLGFYVTLVVNRWWNQFVNLPWPDRLMFLISSSVHGSDEHGRLLRRTLMRYVNLTSLLIFRSVSTAVYKRFPTMDHVVEAGFMTTDERKLFNHLKSPHLKYWVPFIWFGNLATKARNEGRIRDSVDLQSLMTEMNRYRSWCSLLFGYDWVGIPLVYTQVVTLAVYTFFFACLIGRQFLDPTKGYAGHDLDLYIPIFTLLQFFFYAGWLKVAEQLINPFGEDDDDFETNWCIDRNLQVSLLAVDEMHMSLPKMKKDIYWDDSAARPPYTLAAADYCIPSFLGSTVQMGKQMPKNEWKMEDIKIPLPQPQFQCAKSDPGG